(19) United States
(12) Patent Application Publication
(10) Pub. No.: US 2019/0255219 A1
(43) Pub. Date: Aug. 22, 2019

(54) BIOMATERIAL FOR THERAPEUTIC USE

(71) Applicants: ASSISTANCE PUBLIQUE - HÔPITAUX DE PARIS (AP-HP), Paris (FR); INSTITUT NATIONAL DE LA SANTE ET DE LA RECHERCHE MEDICALE (INSERM), Paris (FR); UNIVERSITE RENE DESCARTES-PARIS 5, Paris (FR)

(72) Inventors: Philippe Menasche, Paris (FR); Nisa Renault, Paris (FR); Valerie Bellamy, L' Hay-Les-Roses (FR); Leatitia Pidial, Le Mee Sur Seine (FR)

(21) Appl. No.: 16/331,676

(22) PCT Filed: Sep. 8, 2017
(86) PCT No.: PCT/FR2017/052395

§ 371 Date: Mar. 8, 2019

(30) Foreign Application Priority Data
Sep. 9, 2016 (FR) 1658416

(51) Int. Cl. A61L 27/38; A61L 27/26; A61L 27/24; A61L 27/20

(52) U.S. Cl. CPC A61L 27/3839; A61L 27/26; A61L 2430/20; A61L 27/20; A61L 27/3834; A61L 27/24

(57) ABSTRACT

The present invention relates to a biomaterial for use as medicament intended for the treatment of cardiac tissues, to a process for producing the biomaterial, and also to a biomaterial according to the invention for use as medicament or medical device. The biomaterial of the present invention comprises a biocompatible biodegradable polymer including extracellular vesicles from stem cells. The process of the present invention comprises a step of soaking the biocompatible biodegradable polymer in a biocompatible liquid medium comprising extracellular vesicles from stem cells or from the differentiated derivatives thereof, or a mixing of the biocompatible biodegradable polymer or of the corresponding monomer(s) with extracellular vesicles from stem cells. The biomaterial according to the invention is of use as medicament, especially for the treatment of deficient human or animal cardiac tissue.

### TECHNICAL FIELD

This invention relates to a biomaterial, to a process for producing the biomaterial, as well as to a biomaterial according to the invention, for example for use as medicament or medical device intended for the treatment of cardiac tissues.

This invention has for example applications in the therapeutic field, in humans and in animals, in particular for the treatment of deficient tissues.

The references in brackets “[ ]” hereinbelow refer to the list of references at the end of the examples.

### PRIOR ART

Cell therapy has rapidly shown to be an effective means and with a high potential for restoring deficient tissues caused by a disease, an accident, a genetic mutation, defective or inoperative cell functions, etc. It consists in grafting so called “therapeutic” cells in order restore the function of a tissue or of an organ in a patient. The therapeutic cells used are in fact cells obtained from pluripotent or multipotent stem cells that can come from the patient himself or from a donor. The objective of cell therapy is to treat the patient preferably using an injection of these “therapeutic cells” in order to obtain tissue restoration results that are stable over time.

The publication of Pr. Philippe Menasché et al., “Towards a clinical use of human embryonic stem cell-derived cardiac progenitors: a translational experience”, European Society of Cardiology, European Heart Journal (2015) 36, 743-750 [1] describes for example a set of studies and the results obtained via a cell therapy technique aimed at the heart on rat and non-human primate models having a myocardial infarction. The cells used are cardiac progenitor cells coming from human embryonic stem cells.

Research on stem cells is still controversial, in particular for ethical questions, although it proposes new therapies that are very useful for humans. Additional research is still required in order to improve the current cell therapy techniques, in particular in order to find or perfect in particular the current means for generating, culturing and/or transforming the cells used, and to find means that allow for a single injection or a grafting in the patient, despite the cellular environment and the natural rejection or degradation mechanisms. It is also necessary to comply with the legal framework that is generally restrictive imposed by the States for this type of research. Furthermore, the costs involved with this type of therapy are still very substantial.

It is currently considered that one of the presumed mechanisms of action of grafted stem cells would be a paracrine activation of endogenous signalling pathways. Several studies suggest that this paracrine interaction between grafted cells and host tissue would not be limited to solely the secretion of soluble factors in the extracellular medium, but would involve vesicles coming from cells. Recent work has been published on this subject, in particular in Ana\ddot{l}s Kervadec, Pr. Philippe Menasché et al. “Cardiovascular progenitor-derived extracellular vesicles recapitulate the beneficial effect of their parent cells in the treatment of chronic heart failure”, Elsevier, Original Pre-clinic Science, The Journal of Heart and Lung Transplantation, Jan. 13, 2016; 35:795-807 [2]. This document mentions that extracellular vesicles, including exosomes and microparticles secreted by transplanted cells, could have a paracrine therapeutic effect.

### Exposure of the Invention

This invention has in particular for purpose to resolve the aforementioned disadvantages of prior art by providing an alternative to the use of stem cells for the treating and/or restoring of deficient tissues, in particular de cardiac tissues, in humans and in animals, while still benefiting from the advantages provided by cell therapy, without the aforementioned disadvantages.

The inventors of this invention are in fact the very first to have defined and implemented that combinations of biocompatible polymer(s) and of extracellular vesicles from stem cells make it possible to restore deficient tissues, in particular cardiac tissues, this in the absence of stem cells.

Thus, this invention in particular relates to a biomaterial comprising a biocompatible biodegradable polymer including extracellular vesicles from stem cells.

The inventors of this invention have indeed in particular determined, in the framework of this invention, in particular in the aforementioned combination, that the biomaterial is capable of disappearing progressively once implanted in the organism, in order to ensure a release that is stable and extended in time of the vesicles included in the latter, that does not harm the activity of the vesicles included in the latter, and which allows for an extended treatment of deficient tissues that is economical, free of ethical questions, that is effective and that does not require any new special intervention for removing or replacing implanted equipment.

According to the invention, the term “polymer” means an organic or inorganic polymer or an organic matrix, in particular decellularised cellular, or inorganic or an organic or inorganic polymer and organic or inorganic matrix mixture, the polymer able itself to be considered as a matrix in the framework of this invention since it is advantageously capable of including and progressively releasing vesicles of stem cells, in particular for the treatment of a deficient tissue in a patient, human or animal. Example of polymers that can be used for the implementation of this invention are presented hereinbelow.

According to the invention, the term “biocompatible” polymer means a polymer which is advantageously both compatible for implantation in a patient, i.e. this implantation has a favourable benefit/risk ratio from a therapeutic standpoint, for example in terms of Directive 2001/83/EC, i.e. a risk that is reduced and even non-existent for the patient, versus the therapeutic benefit concerned; and compatible to include therein vesicles of stem cells, i.e. which allows for the inclusion of vesicles, which does not degrade or hardly degrades the activity of the vesicles included in the polymer and/or the biocompatible matrix, and which releases said vesicles once the biomaterial is implanted in a patient, human or animal.

According to the invention, the term “biodegradable” polymer means bioresorbable and/or biodegradable and/or bioabsorbable, with a common purpose of progressive disappearance and, doing this, the progressive release of the vesicles included in the biocompatible polymer, with one or several different or complementary mechanisms for the dissolution or absorption of the polymer in the patient, human or animal, in which the material was implanted. Thus, according to the choice of the polymer among those determined by the inventors as being suitable for implementing this invention, the dissolution can be linked to the material itself which progressively dissolves and/or linked to elimination mechanisms of the human or animal body, in particular through enzymes present in the body fluids.

According to the invention, the biocompatible biodegradable polymer determined by the inventors for the implementation of this invention, can advantageously be:
a polymer of natural origin chosen from the group comprising fibrin, chitosan, collagen, alginate, hyaluronic acid, and a mixture of two or several of the latter;
a matrix, for example a decellularised extracellular matrix;
a synthetic polymer chosen from aliphatic polyesters, in particular polymers derived from lactic acid, in particular a poly(lactic acid), or a poly(glycolic acid), a poly(lactic acid-co-glycolic acid), a poly-(malic acid), in particular poly(beta-malic acid), a polycaprolactone, a polyglycerol sebacate, a polyethylene glycol, a polyurethane or a poly(N-isopropylacrylamide), a mixture of two or several of these aliphatic polyesters, a copolymer of aliphatic polyesters or a mixture of these copolymers, or
a mixture of one or several polymers of natural origin chosen from those mentioned hereinabove and of one or several synthetic polymers chosen from those mentioned hereinabove.

According to the invention, preferably, the biocompatible biodegradable polymer is a polymer of natural origin chosen from the group comprising fibrin, collagen and hyaluronic acid.

These polymers allow indeed for the implementing of this invention and furthermore have the advantages of being, in particular with regards to cardiac tissues, cytocompatible, of making it possible to restore the physiological constraints of the tissue treated, of avoiding a re-operation after the restoring and of allowing a correct restoring of the deficient tissue.

Irrespective of the polymer chosen from those designated in this document, they are preferably of a quality that is sufficient to be implantable and/or injectable into a human or animal organism, in or on a deficient or possibly deficient tissue to be treated, in particular cardiac tissues, without having any risk from a chemical or biological standpoint, in particular microbial contamination, for the organism.

According to the invention, for the implementation of this invention, irrespective of the polymer chosen, for example from fibrin, collagen and hyaluronic acid, it advantageously has a pore size ranging from 50 nm to 1.2 micrometres, preferably from 60 nm to 1 micrometre. This pore size advantageously allows for a sufficient inclusion of vesicles, and a degradation speed of the polymer combined with a release of vesicles that is compatible with the restoration speed of the tissue and/or of its functions.

Fibrin, advantageous in particular for the implementation of this invention for the purpose of treating cardiac tissues, is naturally manufactured during the mechanism of blood coagulation, is generally obtained in a laboratory via a mixture of fibrinogen and thrombin. The thrombin acts as a serine protease that converts the soluble fibrinogen into strands of insoluble fibrin. According to the invention, the term “fibrin” means any form of fibrin. Non-limiting examples of fibrin include fibrin I, fibrin II and fibrin BB or a mixture of these fibrins. Fibrin, when it is formed, forms a network of fibrils separated by pores of which the size can be controlled precisely by adjusting the respective concentrations of fibrinogen and of thrombin, as shown in the examples hereinbelow. The fibrin can be in monomer or polymer form, wherein the polymer form is preferably cross-linked or partially cross-linked. For the implementation of this invention, it has the advantage of being able to be injected, with polymerisation or secondary gelation in situ and/or implanted or deposited in the form of a piece or “patch” on or in a deficient tissue to be treated or on the pathological zone, and to break down naturally once it is implanted. Example of methods and compositions that use a fibrin monomer for preparing fibrin polymers that can be used for the implementation of this invention are described for example, in the documents U.S. Pat. No. 5,750,657 [3], U.S. Pat. No. 5,770,194 [4], U.S. Pat. No. 5,773,418 [5] and U.S. Pat. No. 5,804,428 [6] or in the documents Wong et al., “Fibrin-based biomaterials to deliver human growth factors”, Thromb Haemost 2003; 89:573-82 [7]; Spicer et al. Fibrin glue as a drug delivery system. J Control Release 2010; 148:49-55 [8]; Whelan et al., “Fibrin as a delivery system in wound healing tissue engineering applications”, J Control Release 2014; 196:1-8 [9]; Ahmad et al. Fibrin matrices: “The versatile therapeutic delivery systems”, International Journal of Biomolecules 2015; 81:121-36 [10]. The polymerisation speed of the fibrinogen can be controlled by working with the pH, for example by using pH buffer solutions, and/or by a precise adjustment of the ratio of the respective concentrations of fibrinogen and of thrombin. Examples of products available off the shelf making it possible to obtain fibrin that can be used for the implementation of this invention are preferably of a quality that is sufficient to be implanted and/or injected in or on a tissue of a human or animal organism. This can be for example EVICEL (registered trademark), TISSUCOL KIT (registered trademark), ARTISS (registered trademark), TISSEEL (registered trademark), VIVOSTAT (registered trademark). The inventors have shown for example that a fibrinogen/thrombin concentration ratio of 20 mg/mL-4 U, with the dose of thrombin being distributed homogeneously in the form of droplets of 25-μL, was optimal for obtaining in 5 to 10 minutes at 37° C. the polymerisation of the mixture and the vectorisation of progenitor cardiac cells derived from embryonic stem cells initially mixed with the solution of fibrinogen, as reported in the documents Bellamy et al., “Long-term functional benefits of human embryonic stem cell-derived cardiac progenitors embedded into a fibrin scaffold”, J Heart Lung Transplant. 2015 September; 34:1198-207 [11], and Pr. Philippe Menasché et al., “Towards a clinical use of human embryonic stem cell-derived cardiac progenitors: a translational experience”, Eur Heart J. 2015; 36:743-50 [12].

Chitosan is a polyoside comprised of the random distribution of D-glucosamine bound by ß- and of N-acetyl-D-glucosamine, obtained for example by deacetylation of chitin. It is available off the shelf with a large variety of molecular weights and has a degree of deacetylation between 70% and 90% in general. The molecular weight of chitosan varies from 3,800 to 20,000 Da. Chitosan has structural characteristics similar to glycosaminoglycans (GAG) and seems to imitate their function as described in the document T. Chandy and CP Sharma, “chitosane comme biomatériau”, Biomat, Art. Cells, Art. Org, 18; 1-24 1990 [13]. Examples of chitosane available off the shelf and that can be used for the implementation of this invention can be for example that of lot no. SLBG1673V from Sigma Aldrich, 190 kD, viscosity 20-300 cps, with a degree of acetylation from 75 to 85% or haemostatic agents with a chitosan base currently in clinical use (ChitoFlex (registered trademark), ChitoGauze (registered trademark) PRO.

The collagen that can be used for the implementation of this invention, in particular for the implementation of this invention for the purpose of treating cardiac tissues, is preferably medical collagen coming for example from young herds of cattle or pigs or from free animals that are certified ESB-free (Mad cow disease). The animals of distributors are preferably from closed herds or from countries that have never had any reported cases of BSE such as Australia, Brazil and New Zealand. There is a substantial amount of work that shows that according to the method for producing, collagen or its denatured form, gelatine, can be used as effective vectors of substances in the form of an injectable solution, as described in the document Ladage et al., “Stimulating myocardial regeneration with periostin Peptide in large mammals improves function post-myocardial infarction but increases myocardial fibrosis”, PLoS One. 2013; 8:e59656 [14] or a patch that can have the form of a haemostatic sponge, such as Gelfoam (trademark) as described in the document Polizzoti et al. “Intrapericardial delivery of gelfoam enables the targeted delivery of Periostin peptide after myocardial infarction by inducing fibrin clot formation”, PLoS One, 2012; 7:e36788 [15] or Morcuende et al., “Neuroprotective effects of NGF, BDNF, NT-3 and GDNF on axotomized extraocular motoneurons in neonatal rats”, Neuroscience. 2013; 250:31-48 [16], or a membrane, as described in the documents Wei et al., “Epicardial FSTL1 reconstitution regenerates the adult mammalian heart”, Nature. 2015 Sep. 24; 525(7570):479-85 [17]; Jo et al., “Sequential delivery of BMP-2 and BMP-7 for bone regeneration using a heparinized collagen membrane”, Int J Oral Maxillofac Surg. 2015; 44:921-8.) [18]; and Oliver R. et al, 1976, Clin. Orthop 115: 291-30; 1980, Rr J. Exp Chemin 61, 544-549; 1981, Conn Tissue Res 9: 59-62 [19].

Alginate is a naturally-abundant anionic hydrophilic polysaccharide produced in nature, as described in the document Skjak-Braerk, G.; Grasdalen, H.; Smidsrod, 0. Inhomogeneous polysaccharide ionic gels. Carbohydr. Polym. 1989, 10, 31-54 [20]. Examples of alginates that can be used for the implementation of this invention are described for example in the document Skjak-Braerk et al. [20], or in the documents: Kolambkar et al., “An alginate-based hybrid system for growth factor delivery in the functional repair of large bone defects”, Biomaterials 2011; 32:65-74 [21]; Ruvinov et al., “The effects of controlled HGF delivery from an affinity-binding alginate biomaterial on angiogenesis and blood perfusion in a hindlimb ischemia model”, Biomaterials 2010; 31:4573-4582 [22], as well as in the clinical trial report having evaluated an injectable solution of alginate (Algisyl-LVR (registered trademark) reported in the document Mann et al., “One-year follow-up results from AUGMENT-HF: a multicentre randomized controlled clinical trial of the efficacy of left ventricular augmentation with Algisyl in the treatment of heart failure”, Eur J Heart Fail. 2016; 18:314-25 [23].

Hyaluronic acid, also advantageous for the implementation of this invention for the purpose of treating cardiac tissues, is glycosaminoglycan distributed widely among the connective, epithelial and nerve tissues, is found for example, in the vitreous humour and synovial fluid. It is one of the main components of the extracellular matrix. For the implementation of this invention, it is possible for example to use a product described in the documents: Silva et al., “Delivery of LLKKK18 loaded into self-assembling hyaluronic acid nanogel for tuberculosis treatment”, J Control Release. 2016 Aug. 10; 235:112-[24] or Soranno et al., “Delivery of interleukin-10 via injectable hydrogels improves renal outcomes and reduces systemic inflammation following ischemic acute kidney injury in mice” Am J Physiol Renal Physiol. 2016; 311:F362-72 [25] or Eckhouse et al., “Local hydrogel release of recombinant TIMP-3 attenuates adverse left ventricular remodelling after experimental myocardial infarction”, Sci Transl Med. 2014; 6:223ra21 [26]. This can also be for example a product intended for cutaneous repair, for example Hyalomatrix (registered trademark), as described in the document Landi et al., “Hyaluronic acid scaffold for skin defects in congenital syndactyly release surgery: a novel technique based on the regenerative model”, J Hand Surg Eur Vol. 2014 November; 39(9):994-1000 [27] or a filler product used in cosmetic surgery, such as those presented in the document Gutowski K A., “Hyaluronic acid fillers: Science and clinical uses”, Clin Plast Surg. 2016; 43:489-96 [28]. Hyaluronic acid has the advantage of being able to be injected into the cardiac tissue, for example by using a catheter, or of being delivered via a surgical operation, thoracotomy), in order to form an implant in the form of gel, with this allowing for the controlled delivery of the vesicles over a period for example from 8 to 10 days.

This invention can also be implemented by using a decellularised extracellular matrix. The extracellular matrix is a complex structural entity surrounding and supporting the cells in the tissues of mammals. It is comprised of three major classes of biomolecules: structural proteins, for example, collagen and elastin, specialised proteins, for example, fibrillin, fibronectin and laminin, and proteoglycans, for example, glycosaminoglycans. More preferably, the decellularised extracellular matrix is prepared in such way that the structure of the extracellular matrix is maintained after having been decellularised. It is possible for example to use the protocol described in the document WO2007025233 [29] or in the publication of Khorramirouz R et al., “Effect of three decellularisation protocols on the mechanical behaviour and structural properties of sheep aortic valve conduits”, Adv Med Sci. 2014; 59:299-307 [30] or of He. et al., “Optimization of SDS exposure on preservation of ECM characteristics in whole organ decellularization of rat kidneys”, J Biomed Mater Res B Appl Biomater. 2016 Apr. 8. doi: 10.1002/jbm.b.33668 [31]. for preparing such a matrix, for example from a tissue that preferably is a tissue that corresponds to, or is compatible with, the deficient tissue that has to be treated with the biomaterial of this invention. The matrix can be prepared from an allogeneic or xenogeneic tissue. This can be for example cardiac tissues, such as the decellularised extracellular matrix coming from Ventrigel (trademark) pig hearts described for example in the document Seif-Naraghi et al. “Safety and efficacy of an injectable extracellular matrix hydrogel for treating myocardial infarction”, Sci Transl Med 2013; 5:173ra25 [32].

According to the invention, the polymer can be synthetic, for example chosen from aliphatic polyesters, in particular polymers derived from lactic acid, in particular a poly(lactic acid) or “PLA”, or a poly(glycolic acid), a poly(glycolic acid), a poly(lactic acid-co-glycolic acid), a poly-(malic acid), a polycaprolactone, a mixture of two or several of these aliphatic polyesters, a copolymer of aliphatic polyesters or a mixture of these copolymers.

PLA is an entirely biodegradable polymer wherein the long filamentary molecules are constructed via reaction of an acid group of a molecule of lactic acid on the hydroxyl group with another in order to produce an ester junction. In the body, once implanted, the reaction takes place in the opposite direction and the lactic acid thus released is incorporated into the normal metabolic process. It is possible to increase its speed of biodegradation in the organism, for example by sterilising the polymer via an aggressive method, by introducing into the polymer acid functions or hydrophilic substances. It is also used in surgery where the stitches are carried out with biodegradable polymers which are broken down by reaction with water or under the action of enzymes. This is a material that can also be used by certain 3D printers. Two main methods of synthesis are used to obtain PLA: polycondensation or ring-opening polymerisation. Two monomers are used: (L)-lactic acid (LLA) and (D)-lactic acid (DLLA). PLAs that can be used for the implementation of this invention are for example described in the document Mohiti-Asli and al.; Ibuprofen loaded PLA nanofibrous scaffolds increase proliferation of human skin cells in vitro and promote healing of full thickness incision wounds in vivo. J Biomed Mater Res B Appl Biomater. 2015 Oct. 28. doi: 10.1002/jbm.b.33520 [33]; Tyler et al., “Polylactic acid (PLA) controlled delivery carriers for biomedical applications”, Adv Drug Deliv Rev. 2016 Jul. 15. pii: S0169-409X(16)30211-3. doi: 10.1016/j.addr.2016.06.018 [34]; and James et al., “Poly(lactic acid) for delivery of bioactive macromolecules” Adv Drug Deliv Rev. 2016 Jun. 25. pii: S0169-409X(16)30193-4. doi: 10.1016/j.addr.2016.06.009 [35] or for example available commercially under the brand Resomer (registered trademark—EVONIK company).

Poly(lactic acid-co-glycolic acids) that can be used for the implementation of this invention are for example described in the document Liu et al. “HB-EGF embedded in PGA/PLLA scaffolds via subcritical CO₂ augments the production of tissue engineered intestine. Biomaterials”, 2016; 103:150-9 [36]; Thatcher and al.; “Thymosin β4 sustained release from poly(lactide-co-glycolide) microspheres: synthesis and implications for treatment of myocardial ischemia”, Ann N Y Acad Sci. 2012; 1270:112-9 [37]; or available commercially for example under the brand Resomer (registered trademark).

Poly-(malic acids) that can be used for the implementation of this invention are for example described in the document Portilla-Arias J A et al., “Synthesis, degradability, and drug releasing properties of methyl esters of fungal poly(beta,L-malic acid)”, Macromol Biosci. 2008; 8:540-50 [38]; Loyer et al., “Natural and synthetic poly(malic acid)-based derivates: a family of versatile biopolymers for the design of drug nanocarriers” J Drug Target. 2014; 22:556-75 [39].

Polycaprolactones that can be used for the implementation of this invention are for example described in the document; Patel J J, et al., “Dual delivery of EPO and BMP2 from a novel modular poly-s-caprolactone construct to increase the bone formation in prefabricated bone flaps”, Tissue Eng Part C Methods. 2015; 21:889-97 [40]; Singh S, et al. The enhancement of VEGF-mediated angiogenesis by polycaprolactone scaffolds with surface cross-linked heparin. Biomaterials 2011; 32:2059-2069 [41]; Jeong et al., “Three-dimensional polycaprolactone scaffold-conjugated bone morphogenetic protein-2 promotes cartilage regeneration from primary chondrocytes in vitro and in vivo without accelerated endochondral ossification”, J Biomed Mater Res A. 2012; 100:2088-96 [42]; or are those for example undergoing clinical evaluation in the form of an extemporaneously electro-spun matrix for the treatment of skin wounds, under the reference NCT02680106, Nicast Ltd. company.

According to the invention, it is also possible to use a mixture of one or several polymers of natural origin chosen from those mentioned hereinabove and of one or several synthetic polymers chosen from those mentioned hereinabove.

According to the invention, the term “extracellular vesicles from stem cells”, also called in this document “extracellular vesicles derived from stem cells” or “vesicles” or “microvesicles”, means one or several of the elements produced by stem cells, in particular in culture in vitro, including vesicles or microvesicles, exosomes, apoptotic bodies and microparticles that are found in the environment wherein the stem cells are kept alive. The vesicles in terms of this invention therefore include a heterogeneous population, of which the elements can be differentiated in particular according to their size and their content.

Acting as genuine shuttles, these vesicles play an essential role in intercellular communication by ensuring in particular the transfer of micro-RNA acting on certain key signalling pathways, but also active biolipids and of fragments of DNA capable of gene expression of the cells of the target tissue. The inventors of this invention have in particular validated the functional equivalence of grafted cardiac progenitor cells in the myocardium and of the vesicles that come therefrom. The effective use of these vesicles thanks to the biomaterial of this invention makes it possible to ensure the presence of vesicles in the target tissue for a period that is long enough for them to be able to exert their effects and to prevent a quick elimination from compromising the efficacy of this approach while still causing an undesired systemic dissemination.

According to the invention, the extracellular vesicles from stem cells can come for example from pluripotent, multipotent stem cells or the differentiated derivatives thereof. This can be for example vesicles coming from cells chosen from pluripotent cells, i.e. embryonic stem cells or induced pluripotent somatic cells or induced pluripotent stem cells (iPS), i.e. des cellules taken from the adult and reprogrammed as pluripotent cells by various methods including, but not limited to, adenoviruses, plasmids, transposons, Sendai viruses, synthetic mRNAs and recombinant proteins, for example such as described in the document Takahashi and Yamanaka, “A decade of transcription factor-mediated reprogramming to pluripotency.”, Nat Rev Mol Cell Biol. 2016; 17:183-93 [43]. This can be for example vesicles coming from cells chosen from multipotent cells, for example mesenchymal stem cells, or the differentiated derivatives thereof, and very particularly from cells chosen from cardiac, vascular, muscular, retinal, neural, medullar, osteo-cartilaginous, liver, kidney, intestinal, hematopoietic cells, and cells of the immune system, and very particularly, but not exclusively, dendritic cells. The choice of the cells, and consequently of the vesicles which are used for the implementation of this invention, depends of course on the desired therapeutic target during the use of the biomaterial of this invention, in particular of the deficient tissue to be repaired.

According to the invention, the preparation of vesicles that can be used for the implementation of this invention, from the aforementioned cells has the advantage of not requiring the destruction of the cells and of standardising the final product better. Indeed, the vesicles are secreted by the cells in question in their culture medium of which it was verified beforehand that it does not contain (or contains very little) vesicles which would naturally represent a confounding factor. The extracellular vesicles are taken from these conditioned mediums. The following documents describe methods for culturing stem cells and preparing vesicles coming from these cultures that can be used for the implementation of this invention, from various cell types:
Ana\ddot{l}s Kervadec et al. [2].
Barile et al., “Extracellular vesicles from human cardiac progenitor cells inhibit cardiomyocyte apoptosis and improve cardiac function aftermyocardial infarction”, Cardiovasc Res 2014; 103:530-41 [44].
Bobis-Wozowicz et al., “Human induced pluripotent stem cell-derived microvesicles transmit RNAs and proteins to recipient mature heart cells modulating cell fate and behavior”, Stem Cells. 2015; 33:2748-61 [45].
Lai et al. Exosome secreted by MSC reduces myocardial ischemia/reperfusion injury. Stem cell Res 2012; 4:214-22 [46].
Baulch et al. Cranial grafting of stem cell-derived microvesicles improves cognition and reduces neuropathology in the irradiated brain. Proc Natl Acad Sci USA. 2016; 113:4836-41 [47].
Nojima H and. al—Hepatocyte exosomes mediate liver repair and regeneration via sphingosine-1-phosphate—J Hepatol. 2016 January; 64 (1):60-8 [48].
Herrera M B and. al—Human liver stem cell-derived microvesicles accelerate hepatic regeneration in hepatectomized rats—J Celle Mol Med. 2010 June; 14(GB):1605-18 [49].
Besse B and. al—Dendritic cell-derived exosomes as maintenance immunotherapy after first line chemotherapy in NSCLC—Oncoimmunology. 2015 Aug. 12; 5(4):e1071008. eCollection 2016 [50].
Alessandra Fierabracci and. al—Recent Advances in Mesenchymal Stem Cell Immunomodulation: The Role of Microvesicles—Cell Transplantation, Vol. 24, pp. 133-149, 2015 [51].
Mirjam C. Boelens and. al—Exosome Transfer from Stromal to Breast Cancer Cells Regulates Therapy Resistance Pathways—EMD Millipore Volume 159, Issue 3, p499-513, 23 Oct. 2014 Amnis Imaging Flow Cytometry Integrating flow cytometry and microscopy [52].
Paul D. Robbins and. al—Regulation of Immune Responses by Extracellular Vesicles—Nat Rev Immunol. 2014 March; 14(3): 195-208 [53].
Bin Zhang and. al—Mesenchymal Stem Cells Secrete Immunologically Active Exosomes—Stem Cells and Development, Volume 23, Number 11, 2014 Mary Ann Liebert, Inc. [54].
L Kordelas and. al—MSC-derived exosomes: a novel tool to treat therapy-refractory graft-versus-host disease—Leukemia 2014; 28; 970-3 [55].
Budoni M and. al—The immunosuppressive effect of mesenchymal stromal cells on B lymphocytes is mediated by membrane vesicles—Cell Transplant. 2013; 22(2):369-79 [56].
Christianson H C and. al—Cancer cell exosomes depend on cell-surface heparin sulfate proteoglycans for their internalization and functional activity—Proc Natl Acad Sci USA. 2013 Oct. 22; 110(43):17380-5 [57].
Ratajczak J and. al—Embryonic stem cell-derived microvesicles reprogram hematopoietic progenitors: evidence for horizontal transfer of mRNA and protein delivery—Leukemia. 2006 May; 20(5):847-56 [58].
Morelli A E and. al—The immune regulatory effect of apoptotic cells and exosomes on dendritic cells: its impact on transplantation—Am J Transplant. 2006 February; 6(2):254-61 [59].
Péche H and. al—Presentation of donor major histocompatibility complex antigens by bone marrow dendritic cell-derived exosomes modulates allograft rejection Transplantation. 2003 Nov. 27; 76(10):1503-10 [60].
Celine Akyurekli and. al—A Systematic Review of Preclinical Studies on the Therapeutic Potential of Mesenchymal Stromal Cell-Derived Microvesicles—Stem Cell Rev and Rep, DOI 10.1007/s12015-014-9545-9, 5 Aug. 2014 [61].
Sarah E and. al—Neutrophil-derived microvesicles enter cartilage and protect the joint in inflammatory arthritis—Science Translational Medicine 25 Nov. 2015 Vol 7 Issue 315 315ra190 [62].
Bin Zhang and. al—HucMSC-exosome Mediated—Wnt4 Signaling is Required for Cutaneous Wound Healing—Stem Cells 2014; 00:00-00 AlphaMed Press 2014 [63].
Rekha Nair and. al—Extracellular Vesicles Derived from Preosteoblasts Influence Embryonic Stem Cell Differentiation—Stem Cells and Development Volume 23, Number 14, 2014 Mary Ann Liebert, Inc. [64].
Vincenzo Cantaluppi and. al—Microvesicles Derived From Endothelial Progenitor Cells Enhance Neoangiogenesis of Human Pancreatic Islets—Cell Transplantation. Vol. 21. Pp. 1305-1320. 2012 [65].
Hae Kyung and. al—Mesenchymal stem cells deliver exogenous miRNAs to neural cells and induce their differentiation and glutamate transporter expression—Delivery of miRNAs by MSCs to neural cells. [66]. Stem Cells Dev. 2014; 23:2851-61.
Hongqi Xin and. al—Systemic administration of exosomes released from mesenchymal stromal cells promote functional recovery and neurovascular plasticity after stroke in rats—Journal of Cerebral Blood Flow & Metabolism (2013), 1-5 2013 ISCBFM [67].
Jian-ying Chen and. al—Therapeutic effects of mesenchymal stem cell-derived microvesicles on pulmonary arterial hypertension in rats—Acta Pharmacologica Sinica (4 Aug. 2014) [68].
Lee C and. al—Exosomes mediate the cytoprotective action of mesenchymal stromal cells on hypoxia-induced pulmonary hypertension—Circulation. 2012 Nov. 27; 126(22):2601-11 [69].
Islam M N—Mitochondrial transfer from bone-marrow-derived stromal cells to pulmonary alveoli protects against acute lung injury—Mitochondria to the rescue, [Nat Med. 2012] [70].
Changjin Lee and. al—Exosomes Mediate the Cytoprotective Action of Mesenchymal Stromal Cells on Hypoxia-Induced Pulmonary Hypertension—Circulationaha.112.114173 Published online before print Oct. 31, 2012 [71].
Aliotta J M and. al—Stable cell fate changes in marrow cells induced by lung-derived microvesicles—J Extracell Vesicles. 2012 Apr. 16; 1 [72].
Der-Cherng Targ and. al—Induced Pluripotent Stem Cell-Derived Conditioned Medium Attenuates Acute Kidney Injury by Downregulating the Oxidative Stress-Related Pathway in Ischemia-Reperfusion Rats—Cell Transplantation, Vol. 25, pp. 517-530, 2016 [73].
Bruno S and. al—Microvesicles derived from mesenchymal stem cells enhance survival in a lethal model of acute kidney injury—PloS One. 2012; 7(3):e33115 [74].
Cristina Grange and. al—Biodistribution of mesenchymal stem cell-derived extracellular vesicles in a model of acute kidney injury monitored by optical imaging—International Journal of Molecular Medicine 33: 1055-1063, 2014 [75].
Grange C and. al—Biodistribution of mesenchymal stem cell-derived extracellular vesicles in a model of acute kidney injury monitored by optical imaging—Int J Mol Med. 2014 May; 33(5):1055-63 [76].
Cantaluppi V and. al—Microvesicles derived from endothelial progenitor cells protect the kidney from ischemia-reperfusion injury by microRNA-dependent reprogramming of resident renal cells—Kidney Int. 2012 August; 82(4):412-27 [77].
Vincenzo Cantaluppi and. al—Microvesicles derived from endothelial progenitor cells protect the kidney from ischemia-reperfusion injury by microRNA-dependent reprogramming of resident renal cells—Kidney International advance online publication 11 Apr. 2012 [78].
Joseph V. Bonventre and. al—Microvesicles from Mesenchymal Stromal Cells Protect against Acute Kidney Injury—J Am Soc Nephrol 20: 925-932, 2009 [79].
Stefania Bruno and. al—Mesenchymal Stem Cell-Derived Microvesicles Protect Against Acute Tubular Injury—J Am Soc Nephrol 20: 1053-1067, 2009 [80].
Janet E. and. al—Cranial grafting of stem cell-derived microvesicles improves cognition and reduces neuropathology in the irradiated brain PNAS Early Edition www.pnas.og/cgi/doi/10.1073/pnas.1521668113 [81].
Masako Nakano and. al—Bone marrow-derived mesenchymal stem cells improve diabetes-induced cognitive impairment by exosome transfer into damaged neurons and astrocytes Scientific reports/6:24805/[82].

Thus, the stem cells are kept alive in the culture medium, in vitro, and the vesicles produced by the latter are recovered, advantageously purified, for example as described in the aforementioned documents and/or subjected to a tangential filtration and/or a chromatography, to then implement this invention.

According to the invention, the biocompatible polymer includes the extracellular vesicles from stem cells. The term “includes” means that the polymer is impregnated, like a sponge, with vesicles or that the vesicles are mixed with the polymer in liquid phase and are trapped in the structure of the polymer during the polymerisation thereof. The solution proposed by this invention can thus be considered as a functionalisation or inclusion of the biocompatible polymer chosen with the vesicles chosen to allow for a controlled release of the latter and prevent a fast washing to which a solely the injection of vesicles in an aqueous medium would be exposed. This inclusion or functionalisation can be carried out in particular thanks to the judicious choice of the aforementioned polymers, to the nature of the vesicles chosen and to the technique used to include the vesicles in the polymer.

Inclusion methods in accordance with this invention that can be used for the implementation thereof are described hereinbelow. This inclusion is preferably carried out in a quantity that is sufficient to allow the biomaterial of this invention to release the vesicles all throughout the degradation thereof, and as such maintain, in an extended manner, the treatment of the deficient tissue by the biomaterial. The upper inclusion limit is in particular that linked to inclusion capacity of the polymer chosen. Also, it is useful that the inclusion be sufficient to ensure the extended treatment of the deficient tissue.

The method of the invention is simple and economical. It can be implemented using the polymer chosen or using precursor monomers of the polymer chosen, partially polymerised or not, and of the vesicles chosen.

The method of the invention comprises a step of soaking the biocompatible biodegradable polymer in a biocompatible liquid medium comprising extracellular vesicles from stem cells or from the differentiated derivatives thereof, or from a mixture of the biocompatible biodegradable polymer and/or of the corresponding monomer or monomers, with extracellular vesicles from stem cells or from the differentiated derivatives thereof.

According to the invention, the term “biocompatible liquid medium” means a liquid medium that preserves the vesicles, in their form as well as in their functionality. This is preferably an aqueous solution that sufficiently reproduces the medium wherein the stem cells produce said vesicles, in vitro or in vivo. This can be for example a solution that is close to or identical to the culture medium in vitro of the stem cells chosen to produce the vesicles, or a physiological solution that preserves the vesicles. The culture mediums mentioned in the aforementioned documents are suitable as a biocompatible liquid medium. More preferably, the concentration of the vesicles in the liquid medium is high, so as to allow for a high inclusion of the vesicles in the polymer in order to form the biomaterial of this invention. According to the invention, preferably, the concentration in vesicles is from 3×10¹⁰ to 3×10¹² (30 billion to 3000 billion) vesicles per mL.

In order to reach a preferred concentration, for example from a liquid culture medium of stem cells, it is possible to proceed with the concentration of the vesicles via microfiltration or via centrifugation of the liquid culture medium of the stem cells. Examples of methodologies that can be used to obtain the microvesicles in a biocompatible liquid medium are described in the aforementioned documents [2] and [44] to [82].

According to the invention, the polymer can be manufactured in a first step, then possibly dried or partially dried in such a way as to absorb more vesicles, like a sponge, then impregnated by the vesicles. So also, the precursor monomers of the chosen polymer or the partially cross-linked polymer can be mixed with the chosen vesicles, for example with the biocompatible medium comprising the vesicles, before polymerisation. According to the method of the invention, a polymer that includes the vesicles is then obtained.

According to the invention, the polymer, or its partial monomer or polymer precursor impregnated or mixed with the biocompatible medium comprising the vesicles, can therefore be administered to the patient in injectable form for a polymerisation in situ in the tissue or pathological zone to be repaired, or implanted in the form of a piece or “patch” already polymerised on the tissue or pathological zone to be repaired.

According to the invention, the term “piece” or “patch” means a piece of biomaterial according to the invention, including vesicles, that can be applied on a zone in vivo to be treated, for example a deficient tissue, as defined in this document. This piece or “patch” preferably has a dimension that makes it possible to sufficiently cover, partially or in totality, the surface of the human or animal tissue to be treated, and preferably a thickness that makes it possible to contain a sufficient number of vesicles for the treatment in order to release said vesicles in a prolonged manner. According to the invention, the surface of the piece or “patch” that can be in contact with the tissue to be treated can advantageously have a dimension from 5 to 30 cm², preferably from 15 to 25 cm². According to the invention, the thickness of the piece or “patch” that can be in contact with the tissue to be treated can advantageously be from 0.5 to 2 mm, preferably from 0.8 to 1.2 mm.

For example, according to the invention, advantageously, a piece or a patch that has dimensions such as those mentioned hereinabove, can advantageously comprise from 10¹⁰ to 10¹² vesicles.

According to the invention, monomer or partial polymer such as defined hereinabove can be used as a “glue” to implant the piece or patch on the tissue or on the pathological zone to be repaired, with the polymerisation then allowing for the immobilisation of the patch on the tissue. According to the invention, the immobilisation of the patch on the tissue can also be obtained by gluing the patch on the tissue, or by polymerising at the tissue-patch interface the monomer of the polymer chosen, or by using for example a surgical glue, for example one of the aforementioned polymers can play this role.

The method of this invention can, for example, also be implemented by a technique that makes it possible to precisely define the three-dimensional architecture of the polymer, such as electro-spinning or 3D printing, for example by means of PLA, as described hereinabove. It is possible for example to use the technique described in the document Krishnan et al. “Engineering a growth factor embedded nanofiber matrix niche to promote vascularization for functional cardiac regeneration” Biomaterials. 2016; 97:176-95 [83]; or in the document Lu Y, et al., “Coaxial electrospun fibers: applications in drug delivery and tissue engineering”, Wiley Interdiscip Rev Nanomed Nanobiotechnol. 2016 Feb. 5. doi: 10.1002/wnan.1391. [Epub ahead of print] [84]; or in the document Gaetani R, et al., “Epicardial application of cardiac progenitor cells in a 3D-printed gelatin/hyaluronic acid patch preserves cardiac function after myocardial infarction”, Biomaterials. 2015; 61:339-48 [85].

The biomaterial of this invention makes it possible to treat a deficient tissue without a stem cells graft or as a complement to a stem cells graft.

So, this invention also relates to a biomaterial such as defined in this document, for use as medicament. As disclosed in this document, this can be for example a medicament intended for the treatment of a human or animal deficient tissue. According to the invention, the medicament can be intended for example for the treatment of a tissue chosen from a cardiac, vascular, muscular, ocular, cerebral, medullar, osteo-cartilaginous, liver, kidney, intestinal, hematopoietic or immune tissue.

Among the many advantages already mentioned of this invention, and those appearing to those skilled in the art, it can also be mentioned that the biomaterial of this invention allows for an effective vectorisation of the vesicles delivered in a minimally invasive manner, for example via catheterisation, or surgically and to thus duplicate the productive effect of the stem cells via an a-cell therapy making it possible to overcome many problems of prior art, in particular logistics, cost and ethics, inherent to the transplantation of the cells themselves. According to the invention, the biomaterial can also accompany a cell graft, so as in particular to facilitate engraftment, reduce losses, accompany the treatment of the original tissue and the integration and the functionalisation of the grafted stem cells.

Other characteristics and advantages of this invention can appear to those skilled in the art, in particular in light of the experiments and figures shown hereinbelow for the purposes of information and in a non-limiting way.

### BRIEF DESCRIPTION OF THE FIGURES

FIG. 1 shows cryo-electron microscopy images revealing the presence of the various sub-types of EV

FIG. 2 shows three NTA analyses of three independent preparations of EV showing good reproducibility in the distribution of isolated particle sizes.

FIG. 3 shows a Western Blot confirming the presence of exosome markers and making it possible to show the presence of this vesicular type in the preparation of EV.

FIG. 4 shows photos representing histological sections of patches of fibrin coloured with haematoxylin/eosin

FIG. 5 shows a patch of fibrin with a thickness of 1 mm that can be handled easily and which can be fixed on the epicardium

FIG. 6 shows the effect of the concentration in F/T on the elasticity of the patch in kPa

FIG. 7 shows photographs of control patches and patches containing EVs

FIG. 8 shows an image from ImageStream confirming the release of EV, from the patch, of calcein-marked EV.

FIG. 9 shows the cicatrisation test showing that the EVs of the iPS-Pg are pro-angiogenic

FIG. 10 shows the EVs of iPS-Pg improving the survival of the cardiac cells in culture

FIG. 11 shows the results obtained with ImageStream confirming the presence of fluorescence only in the cellules incubated with the EVs marked beforehand with calcein

### EXAMPLES

### Example 1: Method of Obtained Vesicles from Stem Cells

By way of non-limiting examples, the following protocols are used for the production of vesicles intended for the implementation of this invention:
For the production of vesicles of cardiac cells, the protocol described in the document Barile et al. [44].
For the production of vesicles of mesenchymal stem cells, the protocol described in the document Lai et al. [46].
For the production of vesicles of neural stem cells, the protocol described in the document Baulch et al. [47].

For example in the case of EV of cardiovascular progenitors derived from iPS, cardiac progenitor cells derived from human iPS were used (iPS-Pg: iCell Cardiac Progenitor Cells, ref.: CPC-301-020-001-PT) which come from the supplier Cellular Dynamics International (CDI, Madison, Wis., USA). These progenitors coming from cryopreserved iPS, are defrosted and inoculated at a cell density of 78,000 cells/cm2, in a flask coated beforehand with fibronectin (ref.: 11051407001, Roche Applied Sciences, Indianapolis, Ind., USA) and cultivated for 4 days at 7% CO2 and at 37° C. in a serum-free medium according to the proliferation protocol recommended by the supplier: William's E medium (ref.: A12176-01, Life Technologies, Saint Aubin, France) with the Cocktail B of the pack: “hepatocyte maintenance supplement pack” (ref.: CM4000, Life Technologies), 25 μg/mL of gentamicin (ref.: 15750, Life Technologies) and 1 μg/ml of FGF-2 from Zebrafish (ref.: GFZ1 Cell Guidance System, Cambridge, UK). The extracellular vesicles (EV) are produced and secreted by cells cultivated in serum-free medium. The mediums are changed on the second day of culture (D2) and the conditioned medium is collected on the fourth jour of culture (D4). This conditioned medium contains EVs secreted for 48H (day 3 and day 4). It is recovered for the isolation of the EVs. The mediums and culture conditions could be modified according to the cell type, for example mesenchymal or neural.

Irrespective of the stem cells used, for the isolation of the EVs, the conditioned mediums are recovered and subjected to a conventional centrifugation of 1200 g for 6 min to pellet the cells and contaminating cell debris. The supernatant thus clarified is transferred into a clean tube. It can be frozen at −80° C. in order to store it or used fresh for the isolation of the EVs. For the isolation, the clarified medium is ultracentrifuged in sterile ultracentrifugation tubes at 100,000 g for 16 h, in a vacuum, at a temperature of 4° C. with a maximum acceleration and deceleration. The supernatant after ultracentrifugation is eliminated and the pellet is resuspended in a small volume of sterile PBS, filtered at 0.1 um. The preparation of EV can be used immediately for the implementation of this invention or stored at −80° C.

The vesicles obtained are characterised and analysed by cryo-electron microscopy, via the “Nanoparticle Tracking Analysis” (NTA) technique and via Western blot.

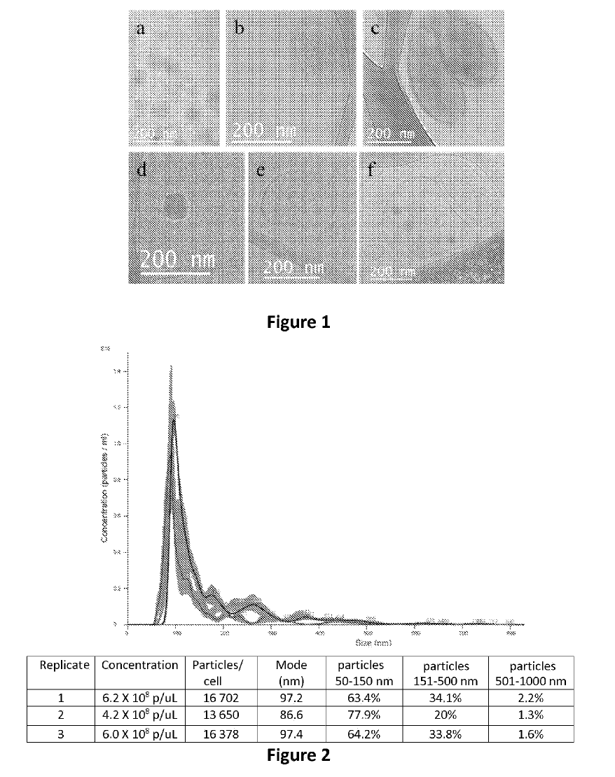

Cryo-electron microscopy makes it possible to view the EVs and to identify the presence of the various sub-populations of EVs, such as the exosomes (50-150 nm), microparticles (100-500 nm) and apoptotic bodies (multivesicular particles >500 nm). Accompanying FIG. 1 shows images (a) to (f) of cryo-electron microscopy revealing the presence of the various sub-types of EV in the case of EVs coming from cardiovascular progenitors derived from iPS. The structures and the size of the exosomes (a, d) and of the microparticles (b, e, c) can be seen. Multivesicular structures (f) which could be apoptotic bodies are also observed.

The “Nanoparticle Tracking Analysis” (NTA) technique, on a Nanosight L M-10 (trademark) platform, with 488 nm laser, (NTA-3.2, Malvern Instruments Ltd., Malvern, UK) makes it possible to determine the distribution of the particle sizes as well as the concentration of EV in each preparation in the case of EVs coming from cardiovascular progenitors derived from iPS. Accompanying FIG. 2 shows three NTA analyses of three independent preparations of EV (replicates 1, 2 and 3) showing good reproducibility in the distribution of isolated particle sizes.

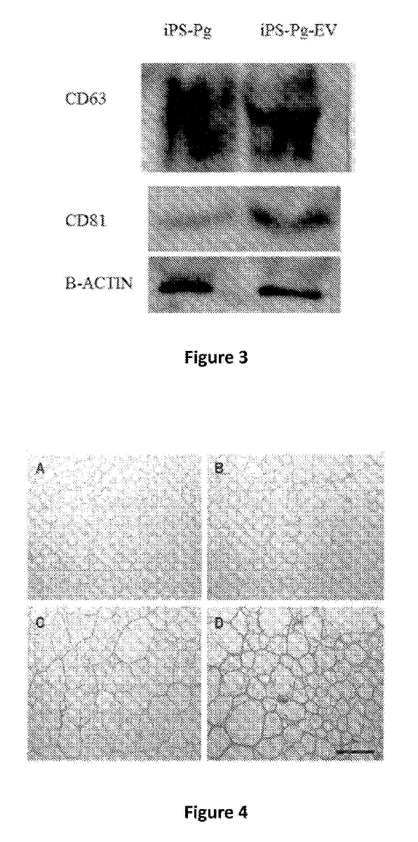

The Western blot technique makes it possible to analyse with antibodies, in particular characteristic CD81 and CD63, the presence of exosomes, sub-type of EV in particular in the case of EVs coming from cardiovascular progenitors derived from iPS. Accompanying FIG. 3 shows the result of this analysis confirming the presence of exosome markers and making it possible to show the presence of this vesicular type in the preparation of EV. The CD81 is enriched in the vesicles with respect to these secreting cells.

### Example 2: Marking Vesicles

In this example, one or the other of the two vesicle marking techniques detailed hereinbelow are used, in order to verify the integration or inclusion of the vesicles in the polymer chosen during the preparation of the biomaterial of this invention, a marking by means of a colouring agent and a marking with calcein-AM (AM=acetoxymethyl):

(a) Marking with a Colouring Agent

To mark the membrane of all of the sub-types of EV obtained, indiscriminately, we use the hydrophobic colouring agent, DiD Vybrant cell tracer (trademark) (ref.: V-22887, Vybrant™ Cell-Labeling Solutions, Molecular Probes), hereinafter “DiD”. The clarified conditioned mediums (see hereinabove) are transferred into ultracentrifugation tubes. The volume is made up with PBS to 15-20 mL. One microlitre of DiD Vybrant cell tracer is added per mL of conditioned and homogenised medium. The conditioned mediums are ultracentrifuged at 100,000 g for 3 h at 4° C. The pellets are resuspended in PBS. The volume is again made up to 20 mL and the suspension is again ultracentrifuged at 100,000 g for 90 min at 4° C. in order to remove the free contaminant DiD. The resulting pellet is resuspended in PBS. The vesicles thus marked are fluorescent in the far red.

(b) Marking of the EVs with Calcein-AM

The protocol described in the document Gray W D et al, Identification of therapeutic covariant microRNA clusters in hypoxia-treated cardiac progenitor cell exosomes using systems biology. Circ. Res. 2015 Jan. 16; 116(2):255-63 [86] is used. The precursor of calcein-AM is a non-fluorescent hydrophobic molecule that passes through the lipid bilayer of the cells and of the EVs. In the presence of functional esterases, the precursor is cleaved, making it fluorescent and hydrophilic, trapping the signal inside the cell. Calcein-AM (cat. C3100MP, Life Technologie) is dissolved in DMSO with a final concentration of 1 mM. The conditioned medium coming from the culture of the iPS-Pg is incubated with 2 μM of calcein AM at 37° C. for 1 h. The mediums are ultracentrifuged for 16 h at 100,000 g. The pellet of marked EV is resuspended in PBS. After having added 17 mL of fresh PBS, the suspension is again ultracentrifuged for 16 h at 100,000 g in order to wash the pellet. The pellet of washed EV is resuspended in clean PBS for the internalisation experiments. The supernatant coming from the washing is used as a control to see the contaminations that are possible by free calcein. The EVs marked with fluorescent calcein in green, and are visible via ImageStream (ImageStream (registered trademark) Mark II Image Flow Cytometer, Merck Millipore), a piece of equipment that allows microscopy imaging to be combined with flow cytometry.

### Example 3: Preparation of Biomaterials According to the Invention

### 3.a) Production of Fibrin Patches:

We have prepared fibrin patches as disclosed in this example. The principle is to resuspend the extracellular vesicles obtained after centrifugation (see example 1 hereinabove) in an aqueous solution of fibrinogen and then to mix this solution with a solution of thrombin. The formation of the fibrin polymer is carried out, then, while coating and trapping the EVs.

The pieces of fibrin or “patches” are prepared using the EVICEL® product (Omrix Biopharmaceutical-Ethicon Biosurgery, Belgium) with the following two components, in order to obtain fibrin:
Human fibrinogen (solution of 2 ml at 50-90 mg/ml)
Human thrombin (solution of 2 ml at 800-1200 U/ml)

The patches are formed using mixtures of a solution of fibrinogen and of a solution of thrombin each dissolved in PBS or an alpha-MEM solution. The ratio of the components is presented in table I hereinbelow, and the ratio of the volumes of the two solutions is set to 1:1. The identical concentrations in the table I hereinbelow (paragraph d)) are prepared using the two aforementioned components, in order to test, in particular, various concentrations of each one of the components and refine the physical characteristics of the patches of fibrin such as the handling capability of the patch, the elasticity of the patch, the size of the mesh and the inclusion and release capacity of the vesicles.

In the figures and in this document, “F” followed by a figure represents the concentration in fibrinogen, with the figure expressing this concentration in mg/mL; and “T”, the concentration in thrombin, with the following expressing this concentration in U/mL.

### 3.b) Characteristics of the Fibrin Patches According to the Concentration of the Components.

Several patches were produced by varying the concentration of fibrinogen/thrombin (F/T). The inventors observed that the higher the concentration in F/T, the faster the patch is polymerised. The main effect of this phenomenon is a variation in the size of the mesh inside the patch: the faster it is polymerised, the larger the pores are. This can be seen easily on cut sections of patches with cryostat and coloured with Hematoxylin/Eosin, as shown in the accompanying FIG. 4. The fibrin fibres appear as a dark violet colour.

In the accompanying FIG. 4, photograph (A) shows a patch with 10 mg/mL of fibrinogen and 2 U/mL thrombin (F10T2); photograph (B) shows a patch with F 20 mg/mL+T 4 U/ml (F20T4); photograph (C) shows a patch with F 40 mg/mL+T 8 U/ml (F40T8). All of the photos are taken with the same magnification. Scale bar: 200 μm.

The photos shown in this figure show that the size of the pores inside the patch varies with the respective concentrations of fibrinogen (F) and thrombin (T), wherein the least concentrated (A) and (B) have pores that are smaller than the most concentrated (C and D). In the pieces or patches produced, a pore size ranging from 50 μm to 200 μm (micrometres) is observed.

A consequence of the variations in the structure of the patch is its handling capability. The inventors have evaluated the handling capability and the suturability of the patches of the various concentrations of F/T.

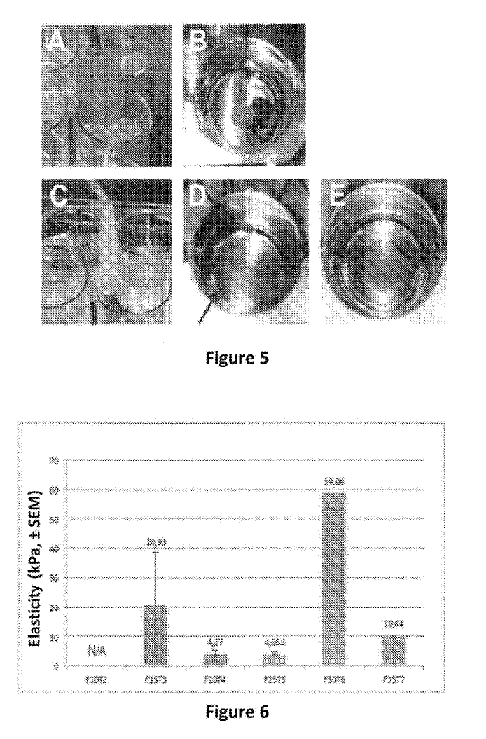

The inventors have observed a handling capacity of the patch that is advantageous for the impregnation of the EVs and the implantation between F5T1 and F40T8. The handling capability of the patch is also according to its thickness. The advantageous thicknesses noted during the experiments are from 0.8 to 1.2 mm. Accompanying FIG. 5 shows by way of example one of the patches obtained, the latter having a thickness of 1 mm.

### 3.d) The Elasticity:

The elasticity of the patches was also considered by the inventors, according to the concentration in F/T. The elasticity is measured directly using the AirExplorerR ultrasound system (Supersonic Imagine) after having selected the region inside the gel. The results, obtained with a Shear Wave Explorer device, show an increase in the rigidity of the patch with the increase in the concentrations in F/T, as shown in accompanying FIG. 6. In this figure is expressed the effect of the concentration in F/T on the elasticity of the patch in kPa. In the case of a cardiac patch, the optimal range in elasticity is between 6 and 15 kPa with a preferred value around 10 kPa. However, the elasticity is to be modulated according to the nature of the target tissue.

### 3.e) Example of an Inclusion Protocol of EVs in a Fibrin Patch of the Size of One Well of a 24-Well Plate.:

### Equipment:

Agarose 5%, sterile
EVs of iPS-Pg prepared according to example 1, taken in sterile PBS
Solution of fibrinogen in sterile PBS (Evicel, Omrix Biopharmaceuticals N.V, Belgium)
Solution of thrombin in sterile PBS (Evicel, Omrix Biopharmaceuticals N.V, Belgium)
Milieu alpha-MEM (ref.: 22571020 GIBCO, Life Technologies)
24-well plate (“P24”) (ref.: 009224, TPP, Dutscher)

### Method:

1/ Prepare agarose at 5%, to be sterilised in the autoclave (liquid protocol at 134° C.)

2/ Deposit this agarose solution still liquid in wells P24 to cover the bottom of the wells and let harden for 20 min. The agarose prevents the fibrin patch from adhering to the bottom of the well.

3/ Choose the concentrations in fibrinogen and in thrombin according to the tableau hereinbelow in order to have the physical characteristics of the desired patch.

4/ Prepare in PBS or alpha-MEM the solution A with the desired concentration of fibrinogen and mixed with 20×10⁶ EV per patch of size P24 in a final volume of 150 μL.

5/ Prepare in PBS or alpha-MEM the solution B with the desired concentration in thrombin in a final volume in 150 μL per patch of size P24.

6/ Thoroughly mix the 150 μL of solution A with the 150 μL of solution B making back-and-forth movements with the pipette in order to correctly homogenize and avoid making bubbles.

7/ Let incubate 20-25 min at 37° C.

8/ Add the alpha-MEM or PBS medium to cover the patch and prevent drying out. About 1 ml of liquid is needed to cover a patch the size of one well of a 24-well plate.

9/ The patches are left to rest, advantageously 4 hours.

TABLE 1
Concentrations of solutions of fibrinogen (F) and
Thrombin (T) for different pieces or patches of fibrin

| | Concentration of | Concentration of |
|---|---|---|
| | fibrinogen (F) in | Thrombin (T) in the |
| | the solution A | solution B |
| F1T0.25 | 1 mg/ml | 0.25 U/ml |
| F2.5T0.5 | 2.5 mg/ml | 0.5 U/ml |
| F5T1 | 5 mg/ml | 1 U/ml |
| F10T2 | 10 mg/ml | 2 U/ml |
| F20T4 | 20 mg/ml | 4 U/ml |
| F40T8 | 40 mg/ml | 8 U/ml |

### 3.f) Visualisation of the Marked EVs in a Fibrin Patch:

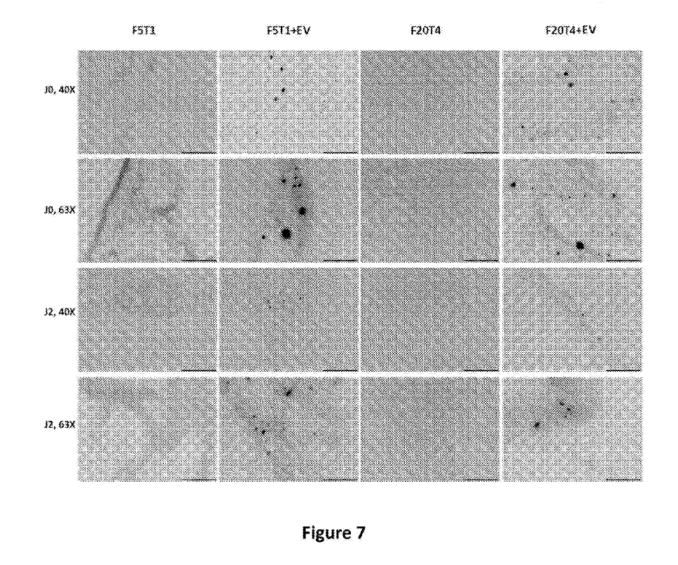

The extracellular vesicles are marked with calcein AM and included in the fibrin patch. This patch is then coated with OCT (trademark) (ref.: TFM-5, MM France), frozen in liquid nitrogen and cryostat cut. The cutting thickness varies between 7 and 10 μm. The sections are viewed using fluorescence microscopy. They are shown in FIG. 7, in the form of photos taken with a magnification of 40× or 63×.

The term “control patch” is used to refer to patches that have not received marked vesicles. The term “EV patch” is used to refer to patches containing extracellular vesicles marked with calcein. FIG. 7 shows the patch containing or not containing extracellular vesicles marked with calcein at two different times of the experiment (D0: immediately after polymerisation; D2: after two days of incubation at 37° C. in the alpha-MEM medium) and at two different magnifications (40× and 63×) on a fluorescence microscope. The lasers have been pushed away on purpose so that we can see the patch for the control patches.

In this figure, the black points correspond to the fluorescent EVs that can be seen inside the patch.

In the control patches, there is no punctiform green fluorescence present which means that there are no vesicles present. The mesh of the patches can be seen in the control patches and the EV patches. However in the “EV patches”, we see the presence of green points in the two patches at the various concentrations used. The presence of these green points means that extracellular vesicles marked with calcein are present. These extracellular vesicles are present inside patches.

### 3.g) Release Study of the EVs of a Fibrin Patch

The patches of the various concentrations of F/T presented hereinabove (table I) containing 20×10⁶ EV marked with calcein-AM are incubated in pure alpha-MEM medium, i.e. a medium containing no particle of the size of the EVs, for 48 hours.

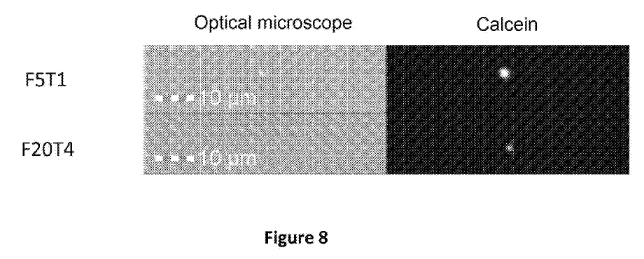

Then, these mediums are collected and centrifuged at 1200 g for 10 min to pellet the fibrin debris, and the supernatant is transferred into a clean tube. The mediums thus clarified are analysed via ImageStream. The results are shown in FIG. 8.

In the conditioned mediums of conditions F5T1+EV and F20T4+EV, the extracellular vesicles were marked and included in the patches. The presence of particles in the conditioned mediums reveals the release of the EVs. This release was confirmed via ImageStream.

The results obtained with ImageStream show that there is indeed a release of the extracellular vesicles in the conditioned medium after 48 h. The comparison of the F5T1+EV and F20T4+EV patches is done with respect to the control which is the medium recovered from the patches F5T1 alone and F20T4 alone. In the mediums of conditions F5T1+EV and F20T4+EV, compared to the negative controls, we observe positive events.

### 3.h) Production of Hyaluronic Acid Patches:

The patch can be the one described in Eckhouse et al. [24]. This can in particular be a product intended for cutaneous repair Hyalomatrix (registered trademark), or a filler product used in cosmetic surgery presented in the document Gutowski K A [25]. Preferably an alpha-MEM or PBS medium is used pour the impregnation with the vesicles, as described hereinabove.

The inventors develop formulations that can be injected into a cardiac tissue by using a catheter (percutaneous approach) or administered surgically (thoracotomy) in order to form implants in the form of a gel that releases the EVs for 8 to 10 days at a controlled rate. The non-cross-linked HA already used for clinical applications is selected.

### 3.i) Production of Collagen Patches:

The patch can be a haemostatic sponge, such as Gelfoam (trademark) as described in the document Polizzoti et al. [15] or of a membrane, as described in the documents Wei et al. [16].

### Example 4: Test of the Efficacy of the Extracellular Vesicles of this Invention for Repairing Deficient Tissues

### a) Analyses of the Bio-Activity of the EVs—Cicatrisation Test (“Wound Healing Assay”):

Vascular endothelial cells coming from human umbilical cord blood (HUVEC single donor, ref: C-12200, PromoCell, Heidelberg, Germany) are inoculated at a density of 42,000 cells/cm² on non-coated 96-well plates and cultivated in a complete medium for endothelial cells (Endothelial Cell Growth Medium, ref: C-22210, PromoCell, Heidelberg, Germany plus Endothelial Cell Growth Medium Supplement Pack, ref:C-39210, PromoCell) at 37° C. and at 5% CO₂ until confluence (all night).

A hole or “scratch zone” or “injury” in then created on a confluent layer of cells at TO (Initial time 0) by scraping it with a pipette tip of 10μl and the scar is located in real time by an inverted microscope (Nikon-ECLIPSE Ti, NIKON CORPORATION, Tokyo, Japan).

The cells are then cultivated for 24 hours in different conditions which are as follows: either in the full medium (positive control), or in the “poor” medium (negative control; medium without complement), or in the poor medium with the isolated EV from pure foetal bovine serum, or in the poor medium with the EVs of iPS-Pg. The images of each well are taken every hours for 24 hours. The surface of the scratch zone is determined at each time by an image analysis.

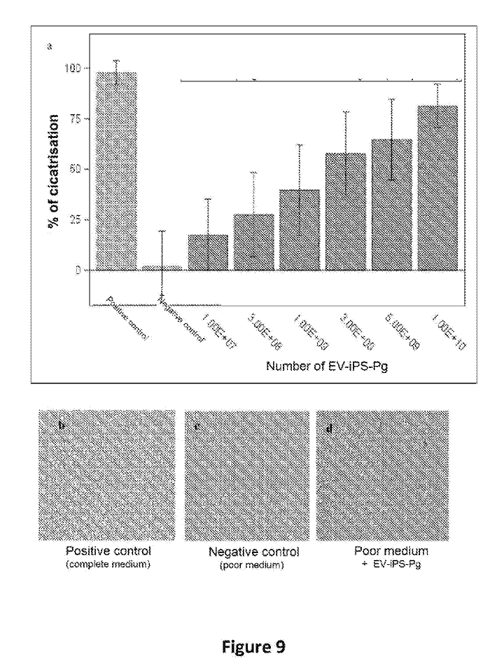

The percentage of cicatrisation is calculated as follows: % cicatrisation=100%×(surface TO—surface T)/(surface TO). Each condition is averaged with three replicates and tested at least on three independent preparations of EV (biological replicates). We have observed that the EVs stimulate the migration of the endothelial cells in a very reproducible way and that the number of vesicles has an effect, as shown in the accompanying FIG. 9. This figure shows the cicatrisation test showing that the EVs of the iPS-Pg are pro-angiogenic:

(a) Histogram showing on the ordinates the percentage of cicatrisation after 18 h of incubation of the HUVEC with different quantities of EV. A dose effect on the migration of the cells is observed.

(b) The injury is entirely healed in the positive control, (c) not healed in the negative control, and (d) almost fully healed at a high dose of EV.

### b) Analyses of the Bio-Activity of the EVc—Viability Test:

An immortalised rat cell line of cardiac myoblasts, H9c2, is inoculated on 6-well plates coated beforehand with 0.2% gelatine at a density of 25,000 cells/cm² and cultivated at 5% CO₂ and at 37° C. in complete medium: DMEM glutamax (ref.: 10566-016, GIBCO, Waltham, Mass., USA), with 10% FBS and 1% Penicillin/Streptomycin/Amphotericin (PSA). After 24 or 48 h, the cells coming from two wells are counted (TO) with the Muse (trademark) Cell Analyzer, according to the instructions of the supplier.

For the other wells, the mediums are changed and the cells are incubated for 27 h in different conditions which are as follows: either in the full medium (positive control), or in the serum-free medium (DMEM glutamax+1% PSA) (negative control, stress medium), or in the serum-free medium with the EVs at different concentrations.

After twenty-seven hours of incubation, the viable cells in each condition are counted with the Muse (trademark) Cell Analyzer (T27). Each condition is evaluated in duplicate and the values obtained are averaged. The percentage of viability is calculated as follows: 100%×(T27 cell count-TO cell count)/TO cell count.

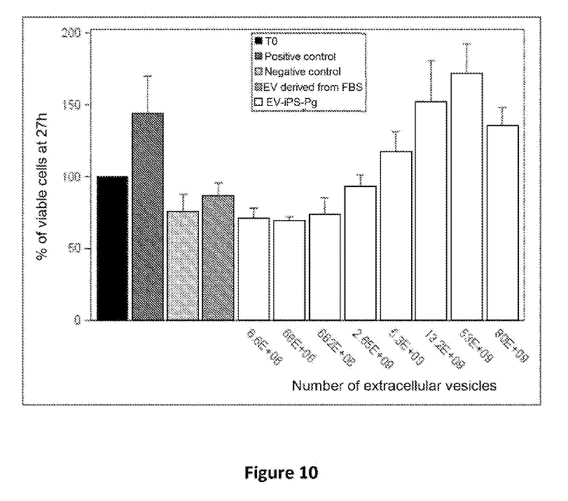

Accompanying FIG. 10 shows the results of three biological replicates (i.e. three different preparations of EV of iPS-Pg evaluated by independent viability test experiments). The EV improves the survival of cardiac cells.

### c) Analyses of the Internalisation of the EVs by Target Cells:

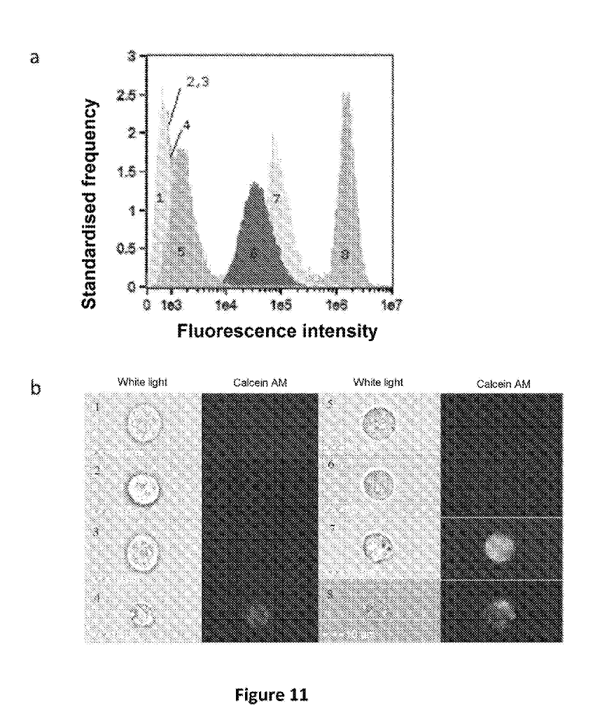

The viability test is used to determine if the EVs marked with calcein AM are internalised. As described hereinabove, the H9c2 are cultivated in a poor medium with or without the addition de EV. Here, the EVs are marked beforehand or not with calcein. After 20 h of incubation, the cells are collected and analysed by the ImageStream. The presence of fluorescence in the H9c2 cells indicates an internalisation of marked EVs. We have observed that the cells incubated with the marked EVs are fluorescent, while the controls are not, as can be seen in accompany FIG. 11.

In this figure, the results obtained with ImageStream confirm the presence of fluorescence only in the cells incubated with the EVs that were marked beforehand with calcein. The fluorescence intensity histograms and the images of the cells cultivated in the presence of calcein AM show a strong green fluorescence in these cells (a-8 and b-7: positive control, cellules+calcein at T19 h, i.e., one hour of marking; a-7 and b-8: cells+calcein for the 20 hours of the experiment). The negative controls without calcein are indeed negative (a-4 and b-1: complete medium; a-3 and b-2 poor medium; a-2 and b-5 EV of iPS-Pg non-marked; a-1 and b-3: EV of FBS non-marked).

The cells incubated with the supernatant after the first washing are slightly more fluorescent than the negative controls (a-5), but this fluorescence is not visible via microscopy (b-6). Finally, the cells incubated with the EV of the iPS-Pg marked with calcein are green with an intensity that is much stronger than with the washing supernatant and less than the cellules marked directly with calcein (a-6 and b-4).

d) Analysis of the Repair of a Deficient Tissue by a Biomaterial in Accordance with this Invention

Patches containing vesicles or not are prepared according to the protocol described in the examples hereinabove. Once manufactured, these patches are deposited or fixed via stitching or glued on the deficient heart.

The evaluation of the efficacy of the patches of vesicles is carried out via echocardiography at different times in order to monitor the change in the cardiac function and the effect compared to the control (i.e. the patch without vesicles).

### Example 5: Preparation of Collagen and Inclusion of the Collagen with Extracellular Vesicles Coming from the Multi or Pluripotent Cells

In this example, we have considered Gelfoam (registered trademark—Pfizer), which is a denatured collagen or gelatine with a high absorption capacity.

With the Gelfoam (registered trademark), patches that are easy to handle and suturable with good elasticities are obtained. The principle of this patch is to incubate it in dry form with a solution of vesicle obtained as described hereinabove so that the latter impregnates the patch with the vesicles (isn't this redundant with the base on page 35, line 30?).

### Example 6: Preparation of EV from Human Umbilical Cord Endothelial Cells

### 1. Preparation of EVs

The human umbilical cord endothelial cells (HUVEC; C-12200; Promocell)) are cultivated in flasks, coated with gelatine 0.2%, at a density of 5000/cm2 in a complete culture medium of endothelial cells (Endothelial Cell Growth Medium; C-22010; Promocell) supplemented with a complement (kit C-39210; Promocell) while will be renewed at D1 and at D4. When the cells are confluent, the complete medium is replaced with a poor medium (medium without supplement). Two days later, the conditioned medium is centrifuged at 1200 g for 6 minutes in order to remove the cellular debris before proceeding with the isolation of the EVs.

The vesicles are marked with two types of markers: DiD (Dialkyl Indocarbocyine Dye) and calcein AM.

i.) Dialkyl Indocarbocyine Dye: The Dialkyl Indocarbocyine (DiD) marker is a lipophilic fluorophore which is internalised by the EVs and emits a fluorescence of which the emission wavelength is 665 nm. The EVs are marked with DiD (V-22887, Vybrant Cell-labeling Solution, Invitrogen) by direct addition of 1 μl of this marker in 1 ml of conditioned medium. The marked medium is stored at 37° C. for one hour before being ultra-centrifuged a first time for the isolation of the EVs then a second time in order to remove the marker that was not internalised. The DiD was used to mark the EVs of FBS because the latter are not marked with calcein AM.

ii.) Acetoxy Methyl calcein: Acetoxy Methyl (AM) calcein is a hydrophobic fluorophore that passes through the bilipid membrane. Once inside the EVs, it is cleaved under the action of an esterase that makes the molecule hydrophilic and fluorescent. Through this mechanism, calcein is a more specific marker for studying EVs than DiD. The calcein AM (cat. C3100MP, Life Technologie) is diluted in 50 μl of DMSO in order to obtain an initial concentration of 1 mM. One volume of this solution of calcein AM is added to the conditioned medium in order to obtain a final concentration of 2 μM. The marked conditioned medium is stored for one hour at 37° C. in order to allow for the marking of the EVs before the step of isolation.

The conditioned mediums of the cells or the foetal bovine serum (FBS), which naturally contain EVs, are subjected to an ultracentrifugation at 37,500 rpm (100,000 g) for 16 hours at 4° C. At the end of the cycle, the supernatant is removed and the pellet is resuspended with PBS filtered at 0.1 μm. When the EVs have been marked beforehand, 20 ml of filtered PBS is added to the resuspended pellets, and a second cycle of ultracentrifugation is run at 37,500 rpm for 16 h at 4° C. in order to remove the fluorophores that were not internalised by the EVs.

### 2. Preparation of Patches

The fibrinogen and the thrombin used for producing the fibrin come from the EVICEL kit (Omrix Biopharmaceuticals-Ethicon Biosurgery, Belgium). For the preparation of the patches, a 24-well plate is prepared 1 hour before the deposition of the fibrinogen thrombin mixture, by depositing 1 ml of sterile agarose at the bottom of each well. This step allows the patch formed to adhere only to the walls of the well without sticking to the bottom in order to facilitate the recovery thereof afterwards.

The patches are formed by a mixture of fibrinogen and thrombin with two different concentrations according to the experiment. Patches prepared with 5 mg/ml of fibrinogen and 1 u/ml of thrombin (F5T1) and patches prepared with 20 mg/ml of fibrinogen and 4 u/ml of thrombin (F20T4). The fibrinogen and the thrombin are diluted in 3 types of different solutions, according to the condition, either alpha-minimal essential medium (αMEM), PBS or NaCl. The fibrinogen and the thrombin are mixed in the 24-well plate, in equal portions i.e. 150 μl each in order to induce polymerisation. After 1 hour at 37° C., the time for the patch to polymerise, 1 ml (or more) of medium (the same as the one that was used for the patch) is added on top of the patch in order to prevent the latter from drying out. The inclusion of the vesicles in the patch is done before polymerisation, by adding the vesicles in the solution of fibrinogen before mixing them with the thrombin.

### 3. Study of the Microscopic Structure of Patches

The fibrin patches are recovered from the wells 4 hours after their preparation, i.e. D0, and 3 days later, i.e. D3. The recovered patches are arranged in wells filled with a cryo-preservation solution (OCT®) and are frozen progressively in liquid nitrogen then stored at −80° C. These patches are cryostat cut in sections 10 μm thick, arranged on a microscope slide that will be frozen in what follows. These slides are coloured with haematoxylin/eosin then analysed with the slide scanner. The analysis of the images thus obtained was carried out using the NDPView software.

### 4. Revealing EVs on Patches

a.) Fluorescence microscope: patches containing EVs of FBS marked with DiD are recovered and frozen at D0 and D3. After the cryostat cutting, the blades are mounted in the fluoroshield and studied with the Leica DM 2000 fluorescence microscope (Leica Wetzlar, Germany) coupled to a Quicam CDD camera (Qimaging corp. Surrey BC Canada). The images were analysed using the Métamorph software.

b.) Confocal microscope: the confocal microscope is an optical microscope that can create images with a very low depth called optical splits. The simple photon confocal system uses an excitation light of which the wavelength directly excites the fluorophore. The confocal system is characterised by a window (or confocal iris) placed in front of the photo-detector that removes the fluorescence coming from the non-focal regions. This system was used to image the EVs on patch with a very high resolution. For this, 3 types of patches were prepared. F5T1 patches containing 21.10⁹ of EV of HUVEC doubly-marked with calcein and with DiD, F5T1 patches with non-marked EVs and bare F5T1 patches without EV, taken as negative controls. These patches are directly recovered on slides adapted to the confocal with Dako® mounting medium and were studied using a confocal microscope of the cell imaging platform of the IMAGINE institute at the Necker hospital directed by Meriem Garfa-Traoré.

c.) Two-photon microscope: Two-photon excitation microscopy is a tool that combines the optical techniques of the confocal microscope with a multi-photon excitation that uses excitation lights in infrared. In this case only the focus point of the laser beam is an exciter. Due to the highly localised excitation, the photo-bleaching of the fluorophores as well as the alteration of the sample are reduced which increases the duration of the experiment. Using an excitation light with a high wavelength (>900 nm) ensures greater penetration inside the sample (up to 500 μm instead of 150 μm) offering the possibility of working on thicker samples. The two-photon microscope allows for the study of the three-dimensional structure of the patch by creating several optical splits without having to section the patch and risk losing material. Three F5T1 patches were prepared for this study. Two patches with or without EV of HUVEC doubly-marked with DiD and with calcein AM and one patch without EV were recovered on slides adapted to the confocal with the mounting medium of the Dako® type and studied using the two-photon microscope of the cell imaging platform of the IMAGINE institute of the Necker hospital.

### 5. Quantification of EVs

a.) Nanoparticle Tracking Analysis: After production and isolation of the EVs, the latter are quantified using Nanoparticle Tracking Analysis (NTA). The NTA or nanosight (LM laser 488 nm, Malvern Instruments Ltd, Malvern, UK) is a method for analysing nanoparticles that allows them to be followed individually. This technology makes it possible to have a concentration and a distribution curve per particle size via dynamic diffusion of the light and analysis of the Brownian motion. NTA comprises an optical microscope that makes it possible to detect the light reflected by the particles suspended in the solution contained in a closed chamber. b.) ImageStream: The ImageStream (ImageStream®X Mark II Imaging Flow Cytometer, Amnis) (IS) is a technology that combines the analysis properties of flow cytometry and microscopic imaging.

This analysis method makes it possible to detect more than different fluorescent markers with a high resolution. This technique requires very little material (15 μl) for the analysis which makes it highly advantageous. This technique makes it possible to view the marked EVs and is used as a control during the marking of the EVs and it also makes it possible to measure the concentration of the marked EVs.

6. Quantification of the Release of the EVs of FBS Marked with DiD

Fibrin patches F5T1 containing 21 10⁹ of EV of FBS marked with DiD were poured into a 24-well plate. After 1 hour of polymerisation, 1 ml of αMEM medium was added and the plate is stored in the incubator at 37° C. The patch and its suspension medium were recovered at different control times, and frozen at −80° C.

The recovered mediums were filtered using a 40 μm screen before the analysis with ImageStream, in order to prevent the obstruction of the capillary of the ImageStream device during the aspiration of the mediums.

7. Quantification of the Release of the EVs of HUVEC Marked with Calcein AM

Fibrin patches F5T1 containing 21 10⁹ of EVs of HUVEC marked with calcein AM were poured into a 24-well plate. One hour after adding the thrombin to the fibrinogen, 1.5 ml of αMEM were added to each well. At each time, the mediums were recovered according to two different schemas:
Change in medium: the medium is recovered completely, i.e. 1.5 ml, and is replaced with an equivalent volume of the αMEM medium.
Aliquot: An aliquot of 250 μl is recovered at each time without adding any additional medium.

The samples are recovered at different control times and are stored either at −80° C. or at 37° C. until they are analysed later.

### 8. Quantification of the Degradation of the Patch

The quantification of the degradation of fibrin patches is carried out via quantification of the D-Dimers with an ELISA D-Dimers kit (DHDDIMER, Human D-DIMER ELISA KIT, Thermo Scientific) according to the protocol of the supplier. The dosage of the D-Dimers is done on the same samples of mediums recovered for the dosage of the EVs marked with calcein so as to compare the release profile of the EVs using the patch with the degradation of the fibrin.

### 9. Handling Capability Tests of the Patches

The purpose of the handling capability tests is to determine the optimum composition of the patch that can be used by the surgeon without difficulties during the operation. An optimum patch is a patch that is easy to recover with conventional tweezers, easily detaches from the walls of the well without tearing, does not shrink after detaching and which is able to return to its initial form once deposited on the tissue during the operation or in a storage solution during tests. Three parameters were chosen for the comparison of the handling of patches:
Taking with tweezers.
Maintaining of the circular shape after removal from the well.
Returning to the initial shape of the patch in the cryoconservation solution.

The fibrin patches were prepared with two different concentrations of fibrinogen and thrombin, F5T1 and F20T4. To these two components, fibrinogen and thrombin, an adequate volume of medium was added. Three different mediums were tested during this experiment, αMEM, PBS and NaCl. These mediums were chosen as they are commonly found in clinical use. The preparation of the patches shows that the latter polymerise and are frozen at different times according to the concentration and the medium added. Indeed, patches F20T4 polymerise faster than the F5T1 patches, and for the same concentration, patches prepared with the αMEM or PBS medium harden faster than patches prepared with NaCl. Four hours after the mixing of the fibrinogen and of the thrombin in the wells, the patches are recovered and placed into small wells with a tissue cryo-preservation gel (OCT®) which will allow them to be frozen.

The fibrin patches, F5T1 and F20T4, prepared with αMEM are easy to recover and to deposit in the wells, retain their circular shape well without folding over onto themselves. The F20T4 patch prepared with PBS has the same characteristics as those obtained with the patches mixed with αMEM, while the F5T1 patches prepared with PBS are more brittle during the detaching from the walls, have a slight retraction when removed from the well, but which disappears after spreading in the cryo-preservation gel.

TABLE I
Results of the handling capability tests of the patches

| | | Taking | Maintaining | |
|---|---|---|---|---|
| | | with | of the | Re- |
| Concentrations | Mediums | tweezers | shape | spreading |
| F5T1 | αMEM | + | + | + |
| | PBS | + | − | + |
| | Na Cl | − | − | − |
| F20T4 | αMEM | + | + | + |
| | PBS | + | + | + |
| | Na Cl | − | − | − |

+: positive result (resistance when taking with tweezers, maintaining of shape, re-spreading)
−: negative result (reduced resistance when taking with tweezers, reduced maintaining of shape, difficult re-spreading)

The F5T1 patches containing the NaCl solution did not succeed in polymerising well. During the recovery of these patches at D0, the texture was not yet rigidified and stuck to the tweezers when removed from the well. One day after the preparation thereof, these patches broke in the wells of the plate.

At the end of these tests, we concluded that the patches prepared with αMEM had the best results in terms of handling capability, in particular for maintaining the structure when removed from the well and the absence of retraction of the polymer thanks to the rigidity of the texture obtained. These characteristics make this batch the best candidate for clinical use. The patches prepared with PBS also show good results.

### 10. Study of the Microscopic Structure of Patches

The fibrin patches are intended to convey EVs that have a size of about a nanometre. The microscopic structure of these patches was studied in order to compare the various architectures obtained with each preparation condition and in order to determine the optimum condition that could work with the addition of the EVs. The various patches were frozen at different times after the polymerisation thereof. The sections obtained with cryostat were coloured with haematoxylin/eosin and digitised with a slide scanner.

The results show that at D0 the F5T1 patches prepared with αMEM or PBS, are not entirely polymerised. Indeed, the study of the microscopic structure of these patches shows a texture of the non-cross-linked fibrin compared to the structure that is viewed, for the same concentration and medium conditions, 3 days later. At D3 the F5T1 patches prepared with αMEM or PBS have an architecture that is well structured, with fine fibres and small pores compared to those obtained with a strong concentration of fibrinogen.

The F20T4 patches, prepared with αMEM or PBS have a similar structure at D0 and at D3. These patches polymerise quickly, as soon as the thrombin is added to the fibrinogen, and organise themselves into a network of fibres with pores that are clearly larger than those viewed with low concentrations. The results show that patches obtained with the NaCl solution of which the structure of the fibres differs from that obtained with the other two mediums. At D0 the F5T1 patch prepared with NaCl has very thick fibres and very large pores that are not homogeneous while the F20T4 patch, prepared with the same solution, has a structure comparable to that observed with the F5T1 patches mixed with αMEM or PBS at D0, therefore a non-polymerised texture. At the end of these results, it seems that the patches prepared with low concentrations of fibrinogen, i.e. F5T1, are the most suitable for conveying the EVs.

The small size of the pores formed by the F5T1 patches after polymerisation seems to be suited to the size of the particles that we want to include therein when they are mixed with the fibrin. In addition, in light of their low concentration, F5T1 patches break down more quickly and therefore drive the release of their content once grafted on the myocardium. The comparison of these results with those obtained during the handling capability tests lead to the conclusion that F5T1 patches mixed with αMEM seem to be the most advantageous polymer for the inclusion of the EVs.

### 11. Revealing EVs on Patches

The purpose of this study was to reveal the presence of EVs on patch polymerised after the mixing thereof in polymer in liquid phase and seeing how these particles are organised inside a fibrin patch knowing that even if these results cannot necessarily be extrapolated as is to other biomaterials, they provide a useful reference base for later developments with different polymers.

a.) Fluorescence microscope: the study of the patches with the fluorescence microscope, shows that the fibrin emits a fluorescence spontaneously in blue. Several red points, of heterogeneous size, can be seen on several positions of the patch only in conditions where EVs marked with DiD were included. These points can be seen only with the red filter and cannot be seen in the control patches where only DiD was added or in the bare patches without marking. These red points could correspond to the EVs of FBS marked with DiD. These particles are localised preferably on fibrin fibres and are rarely viewed on pores of the patch, perhaps because of the rupture of these pores during the cutting of the patches with cryostat which results in a loss of material.

b.) Confocal microscope: F5T1 patches containing EVs of HUVEC doubly-marked with DiD and with calcein AM were analyses, with the confocal microscope, whole without having to cut them with cryostat. The marking of the EVs was controlled with ImageStream before the inclusion thereof into the patch. The study of the patch containing the doubly-marked EVs shows the presence of several fluorescent points of which the size and the colour suggest the EVs included therein. These rounded particles are found disseminated throughout the patch. Certain particles can be seen only in the green filter, others appear only in the red filter and a few particles seem to have internalised the two markers and appear in both filters, greed and red. These fluorescent points are not found in the patches containing the non-marked EVs and in the patches without EV.

c.) Two-photon microscope: the F5T1 patches with EVs doubly-marked with calcein AM and with DiD or without EVs were analysed with the two-photon microscope. The two-photon microscope allows the patch to be examined over its entire thickness, without having to cut it with cryostat or mark the fibrin with a fluophore. The results of the analysis of the patch with the two-photon microscope, show that the fibrin is spontaneously fluorescent in the green filter and the red filter, which makes it possible to distinguish the network that the fibrin forms after the polymerisation thereof. The study of the F5T1 patch containing the EVs marked with calcein and with DiD, shows the presence of several fluorescent points, in green and in red, of different sizes dispersed throughout the patch suggesting the marked EVs. The particles of large size could correspond to several vesicles arranged in a cluster in the polymer. The analysis of the patch in z-stack, shows that these particles are found mostly on fibres of the network and rarely in the cavities of the patch. These fluorescent particles are not found in the control patches, with non-marked EVs and without EV, where only the network of fibrin is viewed.

12. Quantification of the Releasing of the EVs from Patches

For the releasing experiments of the EVs, F5T1 αMEM patches were tested, because the patches obtained at this concentration are not as thick as the F20T4 patches, so this choice of concentration can result in a better diffusion of the EVs of the fibrin network towards the medium and a faster degradation speed of the patches in particular in in-vitro conditions.

a.) Quantification of the Releasing of EVs FBS Marked with DiD

The various mediums are recovered at different times according to the protocol and were stored at −80° C. The day of the analysis, all of the mediums were filtered using a 40 μm screen in order to remove the large debris coming from the degradation of the fibrin which could plug the capillary of the IS device during the aspiration of the mediums. For this experiment, the positive controls are:
qEV DiD+: is a sample containing the equivalent of the dose of EV marked with DiD included in a fibrin patch i.e. 21.10⁹ of particles quantified with NTA (qEV NTA) suspended in 50 μl of PBS filtered at 0.1 μm i.e. a concentration of 4.2.10⁸ particles/μl ([EV]NTA)
PBS DiD+: is a sample of PBS filtered at 0.1 μm marked with a volume of DiD equivalent to the dose required for a marking of EV.
αMEM DiD+: is a sample of αMEM marked with a volume of DiD equivalent to the dose required for a marking of EV.

The negative controls are:
qEV DiD−: is a sample containing 21 10⁹ of non-marked EV.
Filtered PBS: is a sample containing 50 μl of PBS filtered at 0.1 μm.

TABLE II
Results by ImageStream of the control conditions:

| | |
|---|---|
| Samples | Objects/ml DiD+&SSC- |

| | | |
|---|---|---|
| Positive | q EV DiD+ | 1.01E+08 ([EV]_{IS)} |
| controls | 21 × 10⁹ | |
| | PBS DiD+ | 1.99E+06 |
| | MEM DiD+ | 1.64E+06 |
| Negative | q EV DiD− | 3.36E+02 |
| controls | 21 × 10⁹ | |
| | Filtered PBS | 1.22E+03 |
| | alphaMEM | 6.17E+02 |

TABLE III
Results of the quantification by IS of the EVs in
the release mediums of the patches.

| Samples | Times | Objects/ml | qEV DiD+ | Yield |
|---|---|---|---|---|
| F5T1 + EV | D0 | 4.41E+05 | 4.41E+05 | 9% |
| F5T1 + EV | D03 | 1.53E+05 | 1.53E+05 | 3% |
| F5T1 + EV | D07 | 1.45E+05 | 1.45E+05 | 3% |
| F5T1 + EV | D15 | 1.37E+05 | 1.37E+05 | 3% |

The results obtained at IS of the samples of release mediums of the patches are summarised in table III. The yield of each condition is calculated with respect to the yield of the IS for 21 10⁹ EV. This figure is equal to 5.07×10⁶ and it was calculated as follows:

The yield at 100% by IS: ([EV]IS/[EV]NTA)×qEV NTA

Or (1.01×10⁵/4.2×10⁸)×21×10⁹

The results obtained with IS show that the F5T1 patch releases a certain quantity of EV at D0 (about 10% of what was included in the patch). From D0 to D3, a decrease of ⅔ of the quantity of EVs is observed and the yield of the patches passes from 9% to 3%. From D3 to D15 the quantity of EV in the release medium remained constant. This diminution in the EV observed from D0 to D3, can be due to the change in conformation of the patch which passes from a semi-polymerised state at D0 to a fully polymerised and structured state in the days afterward, which could imprison more particles in the polymer during this period via an electrostatic force that would attract the free EVs in the medium towards the inside of the patch. For this analysis, all of the mediums were filtered at 40 μm, this step of filtration which seemed necessary for the correct use of the machine could present a bias for the quantification of the EVs following a loss of a certain quantity of particles that could remain trapped in the fibrin debris removed by the screen. The analyse of the PBS and αMEM mediums containing DiD with IS shows that this marker has the form of suspended particles that can be compared to that viewed with the EVs marked with DiD, which presents a second bias during the quantification of the number of positive events by counting the particles of DiD as being EVs released in the medium. For this reason the choice of the marker for the remainder of the EV quantification experiments has moved to calcein AM.

b.) Quantification of the Release of the EVs of HUVEC Marked with Calcein AM

For this experiment, several conditions were experimented according to the method of recovering the mediums, changing the medium or aliquots, and the storage temperature of the samples, either at −80° C. or at 37° C. Changing the medium of the patches has for purpose to accelerate the phenomenon of diffusion of the particles of the patch towards the medium and to accelerate the phenomenon of the degradation of the fibrin. The storage of the samples at 37° C. is chosen in order to allow for the continuation of the phenomenon of degradation of the fibrin debris recovered in the tubes so as to release a maximum of particles that could remain trapped inside; this would make it possible to overcome the quantification bias of the EVs linked to the step of filtration of the samples before they are analysed with IS.

The comparison of the percentages of release of the EVs by the patch, in the four conditions tested, shows profiles that are rather similar, with a peak in the release over the first 3 days, after the preparation of the patch, then a low release of particles over time. This peak corresponds to the presence of a certain quantity of particles in the mediums recovered at D3, quantified between 10 and 20% of the EVs included in the patch, according to the condition. This peak found again at D3 could correspond to EVs suspended in the αMEM medium which were not fully incorporated into the patch during the polymerisation thereof and were found in suspension when the medium was added. Then the quantity of EVs released by the patch, in the following days is on the average 3 to 5%. This difference in the quantity of EV released by the patches between the first three days and the following days, could be linked to the transformation of the polymer during this period, which passes from a non-cross-linked form, which allows the particles to diffuse easily to a polymerised form that encloses the EVs trapped in the neoformed fibres. In the condition where the medium was aliquoted and stored at 37° C., the quantity of EVs found at D3 is less substantial than that found in the three other conditions; this could be due to a faster polymerisation of this patch or the presence of less EV during the preparation of the latter. The release of the EVs in the following days, is slightly more substantial in the conditions where the medium was fully recovered and changed with fresh medium. This difference in release could be the consequence of the passive diffusion phenomenon of the particles towards the fresh medium added, or a more substantial degradation of the polymer in these conditions.

The storage temperature of the samples does not influence the quantity of EVs found in these mediums, therefore the storage at 37° C. does not seem to drive a more substantial release of the particles that would be trapped in the fibrin debris recovered during the controls.

The comparison of the cumulative release profiles of the EVs from the patches according to the condition with the theoretical passive release curve shows that the release of the EVs is very low and does not follow the change of this curve, therefore the release of the particles from the polymer does not seem linked to the passive diffusion of the latter.

### 13. Monitoring the Degradation of Patches

The same mediums that were analysed with IS in order to quantify the release of the EVs were used for the dosing of the D-Dimers. D-Dimers are products coming from the degradation of the fibrin and the dosage thereof is used as a reference in order to determine if the release of the EVs from patches does indeed depend on the degradation of the polymer or not. The dosage of the D-Dimers shows a peak at D3 in all of the conditions except in the sample recovered by aliquot and stored at 37° C. It is noted that the rate of D-Dimers changes differently in this condition, with absence of a peak at D3 and a progressive increase in the degradation products of the fibrin over time. The comparison of this curve with the dosage of the EVs in the same condition, shows a similar change profile. The quantity of EV in the medium follows the rate of degradation of this patch and the existence of a release peak of the EVs at D3 that is less substantial in this condition than in the other samples tested seems to be linked to the texture of the patch that has a level of degradation that is less substantial during this period than the other patches. In the other conditions, the change in the D-Dimers is comparable amongst themselves. We find a high rate at D3, which corresponds to an increased degradation of these patches, then the quantity of the D-Dimers drops. This could correspond to the moment when the patch changes conformation following the polymerisation and the hardening thereof.

In the conditions where the medium was changed, the rate of D-Dimers decreases until it is cancelled, which suggests a very low or non-existing degradation of these patches. Therefore the yield of the patches in EV, slightly more substantial, in these conditions is linked to a better diffusion of the particles towards the fresh medium added. The comparison of the dosage curves of D-Dimer with the dosage curves of EV in each condition, shows a correlation between the degradation of the polymer and the quantity of the particles found in these mediums. A release peak of the EVs is found in the 3 conditions where a strong rate of d-dimers was dosed. Starting with D7, the concentration of the EV was much less substantial in the conditions where the αMEM medium was changed, therefore those where the degradation of the fibrin is almost non-existing.

### Example 7: Tests In Vivo

In this example, the reinfused myocardial infarction pig model is selected for several reasons: the anatomy of the coronary artery of these species and the shape of the thorax rends it more suitable for the echocardiographic evaluations than sheep and the cardiac size/body weight ratio is close to that of humans. The protocol includes the transfemorative introduction of an angioplasty catheter for an initial view of the network of the coronary artery followed by the insertion of a guide wire that allows for the positioning of an angioplasty balloon in the median portion of the left anterior descending coronary artery (LAD). The balloon is then inflated progressively for 90 minutes in order to interrupt the blood flow in the distal zone of the LAD, downstream of the second major diagonal branch, confirmed by fluoroscopy. After the deflation of the balloon, a second angiogram will be conducted in order to confirm the permeability of the vessel. The prevention of arrhythmias can be provided via a daily oral pre-treatment with amiodarone and beta-blockers and an intra-procedural perfusion of amiodarone. In post-surgery, the animals can receive a daily dose of aspirin and of clopidogrel. Two series of experiments should be conducted in order to imitate the two main clinical situations:

Three weeks after the creation of the infarction, the pigs can undergo a percutaneous endocardial administration of the biomaterial impregnated with EV or of the biomaterial without EV for the controls. After anaesthesia, a guide wire can be advanced into the left ventricle through the aortic valve preceding the insertion of a pigtail catheter on the guide wire. The left ventricular function study can then be conducted in both the anteroposterior and lateral views in order to delimit the regions of myocardial dysfunction. A dedicated catheter (C-Cath®, Celyad, Mont-Saint-Guibert, Belgium) can be inserted through a sheath and advanced to the left ventricle under fluoroscopy. The catheter has a curved 75° needle with lateral holes graduated from small to large size. The gels containing the EVs can be injected at a speed of about 0.5 ml/min into about 10 sites, while waiting a few seconds before removing the needle in order to minimise retrograde leakage.

The analyses of the effect of the administration are conducted 3 months after the injection. The main final point is the functional result evaluated by echocardiography and magnetic resonance imaging (MRI) carried out immediately before the treatment (i.e., 3 weeks after the infarction) and at the time of sacrifice. The MRI sequences will be defined in order to evaluate the LV function (volumes and ejection fraction), myocardial perfusion and the size of the infarction after the injection of gadolinium. After the sacrifice, the explanted hearts are treated for standard histological evaluations (size of the infarction) and immunohistochemical analyses (angiogenesis, infiltration of inflammatory cells, polarisation of macrophages). In addition, blood samples are taken before the treatment and at the time of sacrifice for the analysis of the natriuretic ceramic peptide. The fragments of the myocardium also undergo a transcriptome analysis in order to detect the differential expression of the genes involved in the pathways that contribute to the preservation of the myocardium (survival and proliferation of cells, angiogenesis and vasculogenesis). Finally, biopsies of various organs (lungs, liver, kidneys) are carried out in order to verify the absence of abnormal proliferation of tissues. All of the data will be collected and subject to a blind analysis.

The implant approach in the form of a gel/patch is justified for two reasons. Firstly, certain patients need cardiac surgery after having undergone a myocardial infarction and can benefit from myocardial repair thanks to the material of the invention impregnated with EV, deposited in the form of an implant and/or a gel on the epicardium of the necrosed zone. Thus, another series of experiments, targeted on these surgical applications, can be conducted on the pigs after creation of an infarction according to the interventional procedure described hereinabove. The heart is then approached via left thoracotomy 3 weeks after the creation of the infarction and after visually locating the infarction zone, the material of the invention impregnated with EV is deposited in the form of an implant and/or of a gel on the epicardium of this zone by extending over the limits thereof. The thoracotomy is then closed. Three months later, the animals are euthanized for evaluation according to criteria identical to those described hereinabove concerning treatments carried out by left endoventricular percutaneous administration.

### BIBLIOGRAPHIC REFERENCES

[1] Pr. Philippe Menasché et al.,—Towards a clinical use of human embryonic stem cell-derived cardiac progenitors: a translational experience—European Society of Cardiology, European Heart Journal (2015) 36, 743-750.
[2] Ana\ddot{l}s Kervadec et al.—Cardiovascular progenitor-derived extracellular vesicle recapitulate the beneficial effect of their parent cells in treatment of chronic heart failure—Elsevier, Original Pre-clinic Science, The Journal of Heart and Lung Transplantation, Jan. 13, 2016, 35:795-807.
[3] U.S. Pat. No. 5,750,657—Bristol-Myers Squibb Company.
[4] U.S. Pat. No. 5,770,194—Bristol-Myers Squibb Company.
[5] U.S. Pat. No. 5,773,418—Bristol-Myers Squibb Company.
[6] U.S. Pat. No. 5,804,428—Bristol-Myers Squibb Company.
[7] Wong et al., “Fibrin-based biomaterials to deliver human growth factors”, Thromb Haemost 2003; 89:573-82
[8] Spicer et al. Fibrin glue as a drug delivery system. J Control Release 2010; 148:49-55
[9] Whelan et al., “Fibrin as a delivery system in wound healing tissue engineering applications”, J Control Release 2014; 196:1-8
[10] Ahmad et al. Fibrin matrices: “The versatile therapeutic delivery systems”, International Journal of Biomolecules 2015; 81:121-36.
[11] Bellamy et al., “Long-term functional benefits of human embryonic stem cell-derived cardiac progenitors embedded into a fibrin scaffold”, J Heart Lung Transplant. 2015 September; 34:1198-207.
[12] Pr. Philippe Menasché et al., “Towards a clinical use of human embryonic stem cell-derived cardiac progenitors: a translational experience”, Eur Heart J. 2015; 36:743-50.
[13] T. Chandy and CP Sharma chitosane comme biomatériau Biomat, Art. Cells, Art. Org, 18; 1-24 1990.
[14] Ladage et al., “Stimulating myocardial regeneration with periostin Peptide in large mammals improves function post-myocardial infarction but increases myocardial fibrosis”, PLoS One. 2013; 8:e59656.
[15] Polizzoti et al. “Intrapericardial delivery of gelfoam enables the targeted delivery of Periostin peptide after myocardial infarction by inducing fibrin clot formation”, PLoS One, 2012; 7:e36788.
[16] Morcuende et al., “Neuroprotective effects of NGF, BDNF, NT-3 and GDNF on axotomized extraocular motoneurons in neonatal rats”, Neuroscience. 2013; 250:31-48.
[17] Wei et al., “Epicardial FSTL1 reconstitution regenerates the adult mammalian heart”, Nature. 2015 Sep. 24; 525(7570):479-85.
[18] Jo et al., “Sequential delivery of BMP-2 and BMP-7 for bone regeneration using a heparinized collagen membrane”, Int J Oral Maxillofac Surg. 2015; 44:921-8.).
[19] Oliver R. et al, 1976, Clin. Orthop 115: 291-30; 1980, Rr J. Exp Chemin 61, 544-549; 1981, Conn Tissue Res 9: 59-62.
[20] Skjak-Braerk, G.; Grasdalen, H.; Smidsrod, 0. Inhomogeneous polysaccharide ionic gels. Carbohydr. Polym. 1989, 10, 31-54.
[21] Kolambkar et al., “An alginate-based hybrid system for growth factor delivery in the functional repair of large bone defects”, Biomaterials 2011; 32:65-74.
[22] Ruvinov et al., “The effects of controlled HGF delivery from an affinity-binding alginate biomaterial on angiogenesis and blood perfusion in a hindlimb ischemia model”, Biomaterials 2010; 31:4573-4582.
[23] Mann et al., “One-year follow-up results from AUGMENT-HF: a multicentre randomized controlled clinical trial of the efficacy of left ventricular augmentation with Algisyl in the treatment of heart failure”, Eur J Heart Fail. 2016; 18:314-25.
[24] Silva et al., “Delivery of LLKKK18 loaded into self-assembling hyaluronic acid nanogel for tuberculosis treatment”, J Control Release. 2016 Aug. 10; 235:112-24.
[25] Soranno et al., “Delivery of interleukin-10 via injectable hydrogels improves renal outcomes and reduces systemic inflammation following ischemic acute kidney injury in mice” Am J Physiol Renal Physiol. 2016; 311:F362-72.
[26] Eckhouse et al., “Local hydrogel release of recombinant TIMP-3 attenuates adverse left ventricular remodeling after experimental myocardial infarction”, Sci Transl Med. 2014; 6:223ra21.
[27] Landi et al., “Hyaluronic acid scaffold for skin defects in congenital syndactyly release surgery: a novel technique based on the regenerative model”, J Hand Surg Eur Vol. 2014 November; 39(9):994-1000.
[28] Gutowski K A., “Hyaluronic acid fillers: Science and clinical uses”, Clin Plast Surg. 2016; 43:489-96.
[29] WO2007025233—Regents Of The University Of Minnesota.
[30] Khorramirouz R et al., “Effect of three decellularisation protocols on the mechanical behaviour and structural properties of sheep aortic valve conduits”, Adv Med Sci. 2014; 59:299-307.
[31] He. et al., “Optimization of SDS exposure on preservation of ECM characteristics in whole organ decellularization of rat kidneys”, J Biomed Mater Res B Appl Biomater. 2016 Apr. 8. doi: 10.1002/jbm.b.33668.
[32] Seif-Naraghi et al. “Safety and efficacy of an injectable extracellular matrix hydrogel for treating myocardial infarction”, Sci Transl Med 2013; 5:173ra25.
[33] Mohiti-Asli and al.; Ibuprofen loaded PLA nanofibrous scaffolds increase proliferation of human skin cells in vitro and promote healing of full thickness incision wounds in vivo. J Biomed Mater Res B Appl Biomater. 2015 Oct. 28. doi: 10.1002/jbm.b.33520.
[34] Tyler et al., “Polylactic acid (PLA) controlled delivery carriers for biomedical applications”, Adv Drug Deliv Rev. 2016 Jul. 15. pii: S0169-409X(16)30211-3. doi: 10.1016/j.addr.2016.06.018.
[35] James et al., “Poly(lactic acid) for delivery of bioactive macromolecules” Adv Drug Deliv Rev. 2016 Jun. 25. pii: S0169-409X(16)30193-4. doi: 10.1016/j.addr.2016.06.009.
[36] Liu et al. “HB-EGF embedded in PGA/PLLA scaffolds via subcritical CO₂ augments the production of tissue engineered intestine. Biomaterials”, 2016; 103:150-9.
[37] Thatcher and al.; “Thymosin (34 sustained release from poly(lactide-co-glycolide) microspheres: synthesis and implications for treatment of myocardial ischemia”, Ann N Y Acad Sci. 2012; 1270:112-9.
[38] Arias J A et al., “Synthesis, degradability, and drug releasing properties of methyl esters of fungal poly(beta,L-malic acid)”, Macromol Biosci. 2008; 8:540-50.
[39] Loyer et al., “Natural and synthetic poly(malic acid)-based derivates: a family of versatile biopolymers for the design of drug nanocarriers” J Drug Target. 2014; 22:556-75.
[40] Patel J J, et al., “Dual delivery of EPO and BMP2 from a novel modular poly-s-caprolactone construct to increase the bone formation in prefabricated bone flaps”, Tissue Eng Part C Methods. 2015; 21:889-97.
[41] Singh S, et al. The enhancement of VEGF-mediated angiogenesis by polycaprolactone scaffolds with surface cross-linked heparin. Biomaterials 2011; 32:2059-2069.
[42] Jeong et al., “Three-dimensional polycaprolactone scaffold-conjugated bone morphogenetic protein-2 promotes cartilage regeneration from primary chondrocytes in vitro and in vivo without accelerated endochondral ossification”, J Biomed Mater Res A. 2012; 100:2088-96.
[43] Takahashi and Yamanaka, “A decade of transcription factor-mediated reprogramming to pluripotency.”, Nat Rev Mol Cell Biol. 2016; 17:183-93.
[44] Barile et al., “Extracellular vesicles from human cardiac progenitor cells inhibit cardiomyocyte apoptosis and improve cardiac function aftermyocardial infarction”, Cardiovasc Res 2014; 103:530-41.
[45] Bobis-Wozowicz et al., “Human induced pluripotent stem cell-derived microvesicles transmit RNAs and proteins to recipient mature heart cells modulating cell fate and behavior”, Stem Cells. 2015; 33:2748-61.
[46] Lai et al. Exosome secreted by MSC reduces myocardial ischemia/reperfusion injury. Stem cell Res 2012; 4:214-22.
[47] Baulch et al. Cranial grafting of stem cell-derived microvesicles improves cognition and reduces neuropathology in the irradiated brain. Proc Natl Acad Sci USA. 2016; 113:4836-41.
[48] Nojima H and. al—Hepatocyte exosomes mediate liver repair and regeneration via sphingosine-1-phosphate—J Hepatol. 2016 January; 64(1):60-8.
[49] Herrera M B and. al—Human liver stem cell-derived microvesicles accelerate hepatic regeneration in hepatectomized rats—J Celle Mol Med. 2010 June; 14(GB):1605-18.
[50] Besse B and. al—Dendritic cell-derived exosomes as maintenance immunotherapy after first line chemotherapy in NSCLC—Oncoimmunology. 2015 Aug. 12; 5(4):e1071008. eCollection 2016.
[51] Alessandra Fierabracci and. al—Recent Advances in Mesenchymal Stem Cell Immunomodulation: The Role of Microvesicles—Cell Transplantation, Vol. 24, pp. 133-149, 2015.
[52] Mirjam C. Boelens and. al—Exosome Transfer from Stromal to Breast Cancer Cells Regulates Therapy Resistance Pathways—EMD Millipore Volume 159, Issue 3, p499-513, 23 Oct. 2014 Amnis Imaging Flow Cytometry Integrating flow cytometry and microscopy.
[53] Paul D. Robbins and. al—Regulation of Immune Responses by Extracellular Vesicles—Nat Rev Immunol. 2014 March; 14(3): 195-208.
[54] Bin Zhang and. al—Mesenchymal Stem Cells Secrete Immunologically Active Exosomes—Stem Cells and Development, Volume 23, Number 11, 2014 Mary Ann Liebert, Inc.
[55] L Kordelas and. al—MSC-derived exosomes: a novel tool to treat therapy-refractory graft-versus-host disease—Leukemia 2014; 28; 970-3.
[56] Budoni M and. al—The immunosuppressive effect of mesenchymal stromal cells on B lymphocytes is mediated by membrane vesicles—Cell Transplant. 2013; 22(2):369-79.
[57] Christianson H C and. al—Cancer cell exosomes depend on cell-surface heparin sulfate proteoglycans for their internalization and functional activity—Proc Natl Acad Sci USA. 2013 Oct. 22; 110(43):17380-5.
[58] Ratajczak J and. al—Embryonic stem cell-derived microvesicles reprogram hematopoietic progenitors: evidence for horizontal transfer of mRNA and protein delivery—Leukemia. 2006 May; 20(5):847-56.
[59] Morelli A E and. al—The immune regulatory effect of apoptotic cells and exosomes on dendritic cells: its impact on transplantation—Am J Transplant. 2006 February; 6(2):254-61.
[60] Péche H and. al—Presentation of donor major histocompatibility complex antigens by bone marrow dendritic cell-derived exosomes modulates allograft rejection Transplantation. 2003 Nov. 27; 76(10):1503-10.
[61] Celine Akyurekli and. al—A Systematic Review of Preclinical Studies on the Therapeutic Potential of Mesenchymal Stromal Cell-Derived Microvesicles—Stem Cell Rev and Rep, DOI 10.1007/s12015-014-9545-9, 5 Aug. 2014.
[62] Sarah E and. al—Neutrophil-derived microvesicles enter cartilage and protect the joint in inflammatory arthritis—Science Translational Medicine 25 Nov. 2015 Vol 7 Issue 315 315ra190.
[63] Bin Zhang and. al—HucMSC-exosome Mediated—Wnt4 Signaling is Required for Cutaneous Wound Healing—Stem Cells 2014; 00:00-00 AlphaMed Press 2014.
[64] Rekha Nair and. al—Extracellular Vesicles Derived from Preosteoblasts Influence Embryonic Stem Cell Differentiation—Stem Cells and Development Volume 23, Number 14, 2014 Mary Ann Liebert, Inc.
[65] Vincenzo Cantaluppi and. al—Microvesicles Derived From Endothelial Progenitor Cells Enhance Neoangiogenesis of Human Pancreatic Islets—Cell Transplantation. Vol. 21. Pp. 1305-1320. 2012.
[66] Hae Kyung and. al—Mesenchymal stem cells deliver exogenous miRNAs to neural cells and induce their differentiation and glutamate transporter expression—Delivery of miRNAs by MSCs to neural cells. Stem Cells Dev. 2014; 23:2851-61.
[67] Hongqi Xin and. al—Systemic administration of exosomes released from mesenchymal stromal cells promote functional recovery and neurovascular plasticity after stroke in rats—Journal of Cerebral Blood Flow & Metabolism (2013), 1-5 2013 ISCBFM.
[68] Jian-ying Chen and. al—Therapeutic effects of mesenchymal stem cell-derived microvesicles on pulmonary arterial hypertension in rats—Acta Pharmacologica Sinica, 4 Aug. 2014.
[69] Lee C and. al—Exosomes mediate the cytoprotective action of mesenchymal stromal cells on hypoxia-induced pulmonary hypertension—Circulation. 2012 Nov. 27; 126(22):2601-11.
[70] Islam M N et al.—Mitochondrial transfer from bone-marrow-derived stromal cells to pulmonary alveoli protects against acute lung injury—Mitochondria to the rescue, Nat Med. 2012.
[71] Changjin Lee and. al—Exosomes Mediate the Cytoprotective Action of Mesenchymal Stromal Cells on Hypoxia-Induced Pulmonary Hypertension—Circulationaha.112.114173 Published online before print Oct. 31, 2012.
[72] Aliotta J M and. al—Stable cell fate changes in marrow cells induced by lung-derived microvesicles—J Extracell Vesicles. 2012 Apr. 16; 1.
[73] Der-Cherng Targ and. al—Induced Pluripotent Stem Cell-Derived Conditioned Medium Attenuates Acute Kidney Injury by Downregulating the Oxidative Stress-Related Pathway in Ischemia-Reperfusion Rats—Cell Transplantation, Vol. 25, pp. 517-530, 2016.
[74] Bruno S and. al—Microvesicles derived from mesenchymal stem cells enhance survival in a lethal model of acute kidney injury—PloS One. 2012; 7(3):e33115.
[75] Cristina Grange and. al—Biodistribution of mesenchymal stem cell-derived extracellular vesicles in a model of acute kidney injury monitored by optical imaging—International Journal of Molecular Medicine 33: 1055-1063, 2014.
[76] Grange C and. al—Biodistribution of mesenchymal stem cell-derived extracellular vesicles in a model of acute kidney injury monitored by optical imaging—Int J Mol Med. 2014 May; 33 (5):1055-63.
[77] Cantaluppi V and. al Microvesicles derived from endothelial progenitor cells protect the kidney from ischemia-reperfusion injury by microRNA-dependent reprogramming of resident renal cells—Kidney Int. 2012 August; 82(4):412-27.
[78] Vincenzo Cantaluppi and. al—Microvesicles derived from endothelial progenitor cells protect the kidney from ischemia-reperfusion injury by microRNA-dependent reprogramming of resident renal cells—Kidney International advance online publication 11 Apr. 2012.
[79] Joseph V. Bonventre and. al—Microvesicles from Mesenchymal Stromal Cells Protect against Acute Kidney Injury—J Am Soc Nephrol 20: 925-932, 2009.
[80] Stefania Bruno and. al—Mesenchymal Stem Cell-Derived Microvesicles Protect Against Acute Tubular Injury—J Am Soc Nephrol 20: 1053-1067, 2009.
[81] Janet E. and. al—Cranial grafting of stem cell-derived microvesicles improves cognition and reduces neuropathology in the irradiated brain—PNAS Early Edition www.pnas.og/cgi/doi/10.1073/pnas.1521668113.
[82] Masako Nakano and. al—Bone marrow-derived mesenchymal stem cells improve diabetes-induced cognitive impairment by exosome transfer into damaged neurons and astrocytes Scientific reports/6:24805/.
[83] Krishnan et al. “Engineering a growth factor embedded nanofiber matrix niche to promote vascularization for functional cardiac regeneration” Biomaterials. 2016; 97:176-95.
[84] Lu Y, et al., “Coaxial electrospun fibers: applications in drug delivery and tissue engineering”, Wiley Interdiscip Rev Nanomed Nanobiotechnol. 2016 Feb. 5. doi: 10.1002/wnan.1391. [Epub ahead of print].
[85] Gaetani R, et al., “Epicardial application of cardiac progenitor cells in a 3D-printed gelatin/hyaluronic acid patch preserves cardiac function after myocardial infarction”, Biomaterials. 2015; 61:339-48.
[86] Gray W D et al, Identification of therapeutic covariant microRNA clusters in hypoxia-treated cardiac progenitor cell exosomes using systems biology. Circ. Res. 2015 Jan. 16; 116(2):255-63.

1. A method of treating cardiac tissues, the method comprising administering a biomaterial comprising a biocompatible biodegradable polymer including extracellular vesicles from a stem cells.

2. The method according to claim 1, wherein the extracellular vesicles from the stem cell is derived from stem cells selected from the group consisting of pluripotent stem cells, multipotent stem cells or the differentiated derivatives thereof.

3. The method according to claim 2, wherein the extracellular vesicles come from cells selected from the group consisting of cardiac cells, and vascular cells.

4. The method according to claim 1, wherein the biocompatible biodegradable polymer is a polymer of natural origin including at least one of:
(i) fibrin, chitosan, collagen, alginate, hyaluronic acid and mixtures thereof;
(ii) a synthetic polymer, wherein the polymers are selected from aliphatic polyesters; or
(iii) a decellularized extracellular matrix.

5. The method according to claim 1, wherein the biocompatible biodegradable polymer is a polymer of natural origin chosen from the group consisting of fibrin, collagen and hyaluronic acid.

6. The method according to claim 1, wherein the biomaterial is obtained by a method comprising:
soaking the biocompatible biodegradable polymer in a biocompatible liquid medium comprising extracellular vesicles from the stem cell or from the differentiated derivatives thereof; or
mixing the biocompatible biodegradable polymer and/or the corresponding monomer or monomers with extracellular vesicles from the stem cell or from the differentiated derivatives thereof.

7. A method for producing a biomaterial comprising a biocompatible biodegradable polymer including extracellular vesicles from a stem cell, the method comprising:
soaking the biocompatible biodegradable polymer in a biocompatible liquid medium comprising extracellular vesicles from the stem cell or from the differentiated derivatives thereof; or
mixing the biocompatible biodegradable polymer or the corresponding monomer or monomers with extracellular vesicles from the stem cell or from the differentiated derivatives thereof.

8. The method according to claim 4, wherein the aliphatic polyesters are selected from the group consisting of a poly(lactic acid), a poly(glycolic acid), poly(lactic acid-co-glycolic acid), poly-(malic acid), a polycaprolactone, a polyglycerol sebacate, a polyurethane or a poly(N-isopropylacrylamide), and mixtures thereof.

9. The method according to claim 8, wherein the biocompatible biodegradable polymer is a copolymer of the aliphatic polyesters or a mixture of copolymers of the aliphatic polyesters.

10. The method according to claim 8, wherein the biocompatible biodegradable polymer is a mixture of copolymers of the aliphatic polyesters.

11. The method according to claim 2, wherein the biocompatible biodegradable polymer is a polymer of natural origin selected from the group consisting of:
(i) fibrin, chitosan, collagen, alginate, hyaluronic acid and mixtures thereof;
(ii) a synthetic polymer, wherein the polymers are selected from aliphatic polyesters; or
(iii) a decellularized extracellular matrix.

12. The method according to claim 3, wherein the biocompatible biodegradable polymer is a polymer of natural origin including at least one of:
(i) fibrin, chitosan, collagen, alginate, hyaluronic acid and mixtures thereof;
(ii) a synthetic polymer, wherein the polymers are selected from aliphatic polyesters; or
(iii) a decellularized extracellular matrix.

13. The method according to claim 7, wherein the extracellular vesicles from the stem cell come from stem cells selected from the group consisting of pluripotent stem cells, multipotent stem cells, or the differentiated derivatives thereof.

14. The method according to claim 13, wherein the extracellular vesicles come from cells selected from the group consisting of cardiac cells, and vascular cells.

15. The method according to claim 7, wherein the biocompatible biodegradable polymer is a polymer of natural origin including at least one of:
(i) fibrin, chitosan, collagen, alginate, hyaluronic acid and mixtures thereof;
(ii) a synthetic polymer, wherein the polymers are selected from aliphatic polyesters; or
(iii) a decellularized extracellular matrix.

16. The method according to claim 15, wherein the aliphatic polyesters are selected from the group consisting of a poly(lactic acid), a poly(glycolic acid), poly(lactic acid-co-glycolic acid), poly-(malic acid), a polycaprolactone, a polyglycerol sebacate, a polyurethane or a poly(N-isopropylacrylamide), and mixtures thereof.

17. The method of claim 15, wherein the biocompatible biodegradable polymer is a copolymer of the aliphatic polyesters or a mixture of copolymers of the aliphatic polyesters.

18. The method according to claim 1, wherein the biomaterial is administered within or in apposition to the cardiac tissue or a combination thereof.

19. The method according to claim 18, wherein the biomaterial is administered to at least one of an outer surface, an inner surface, within the cardiac tissue or a combination thereof.

20. The method according to claim 19, wherein the biomaterial is administered by implantation or injection.

21. The method according to claim 1, wherein the extracellular vesicles includes one or more extracellular elements produced by the stem cell.

22. The method according to claim 21, wherein at least one of:
the extracellular element are selected from vesicles or microvesicles, exosomes, apoptotic bodies, and microparticles produced by the stem cell; and
the stem cell are culture in vitro.
